(19) United States
(12) Patent Application Publication
(10) Pub. No.: US 2015/0206727 A1
(43) Pub. Date: Jul. 23, 2015

(54) Method and apparatus for underdetermined blind separation of correlated pure components from nonlinear mixture mass spectra

(71) Applicant: RUDJER BOSKOVIC INSTITUTE, Zagreb (HR)

(72) Inventors: Ivica Kopriva, Zagreb (HR); Ivanka Jeric, Zagreb (HR); Lidija Brkljacic, Zagreb (HR)

(73) Assignee: RUDJER BOSKOVIC INSTITUTE, Zagreb (HR)

(21) Appl. No.: 14/157,578

(22) Filed: Jan. 17, 2014

(51) Int. Cl. H01J 49/00; G01N 33/50; G01N 33/49; H01J 49/26; G01N 33/66

(52) U.S. Cl. CPC H01J 49/0036; H01J 49/26; G01N 33/66; G01N 33/49; G01N 33/5091

(57) ABSTRACT

The present invention relates to a computer-implemented method and apparatus for data processing for the purpose of blind separation of nonnegative correlated pure components from smaller number of nonlinear mixtures of mass spectra. More specific, the invention relates to preprocessing of recorded matrix of mixtures spectra by robust principal component analysis, trimmed thresholding, hard thresholding and soft thresholding; empirical kernel map-based nonlinear mappings of preprocessed matrix of mixtures mass spectra into reproducible kernel Hilbert space and linear sparseness and nonnegativity constrained factorization of mapped matrices therein. Thereby, preprocessing of recorded matrix of mixtures mass spectra is performed to suppress higher order monomials of the pure components that are induced by nonlinear mixtures. Components separated by each factorization are correlated with the ones stored in the library. Thereby, component from the library is associated with the separated component by which it has the highest correlation coefficient. Value of the correlation coefficient indicates degree of pureness of the separated component. Separated components that are not assigned to the pure components from the library can be considered as candidates for new pure components. Identified pure components can be used for identification of compounds in chemical synthesis, food quality inspection or pollution inspection, identification and characterization of compounds obtained from natural sources (microorganisms, plants and animals), or in instrumental diagnostics—determination and identification of metabolites and biomarkers present in biological fluids (urine, blood plasma, cerebrospinal fluid, saliva, amniotic fluid, bile, tears, etc.) or tissue extracts.

### FIELD OF THE INVENTION

The present invention relates to a computer-implemented method and apparatus for data processing for the purpose of blind separation of nonnegative correlated pure components from smaller number of nonlinear mixtures of mass spectra. The invention relates to preprocessing of recorded matrix of mixtures spectra by robust principal component analysis, trimmed thresholding, hard thresholding and soft thresholding; empirical kernel map-based nonlinear mappings of preprocessed matrix of mixtures mass spectra into reproducible kernel Hilbert space and linear sparseness and nonnegativity constrained factorization of mapped matrices therein. Thereby, preprocessing of recorded matrix of mixtures mass spectra is performed to suppress higher order monomials of the pure components that are induced by nonlinear mixtures. Components separated by each factorization are correlated with the ones stored in the library. Thereby, component from the library is associated with the separated component by which it has the highest correlation coefficient. Value of the correlation coefficient indicates degree of pureness of the separated component. Separated components that are not assigned to the pure components from the library can be considered as candidates for new pure components. Identified pure components can be used for identification of compounds in chemical synthesis, food quality inspection or pollution inspection, identification and characterization of compounds obtained from natural sources (microorganisms, plants and animals), or in instrumental diagnostics—determination and identification of metabolites and biomarkers present in biological fluids (urine, blood plasma, cerebrospinal fluid, saliva, amniotic fluid, bile, tears, etc.) or tissue extracts.

### BACKGROUND OF THE INVENTION

Quantification and identification of the pure components present in the mixture is a traditional problem in NMR, IR, UV, EPR and Raman spectroscopy, mass spectrometry, etc. In majority of applications separation of pure components is performed by assuming that mixture spectra are linear combinations of pure components. While linear mixture model applies to many scenarios nonlinear mixture model offers more accurate description of biochemical processes and interactions occurring during perturbations of biological systems caused by disease or drug treatment. Few examples of nonlinear model reactions that mimic biological processes include: esterification of amino acids and short peptides, ester hydrolysis and amide bond formation in the synthesis of peptides.

Identification of pure components present in mixtures proceeds often by matching separated components spectra with a library of reference compounds. Degree of correlation depends on how well pure components present in mixtures spectra are separated from each other. Thereby, of interest are methods for blind separation of pure components from mixtures spectra that use as input information the matrix with recorded mixtures spectra only. There are many methods for blind separation of pure components from mixtures spectra assuming linear mixture model. To name a few, we mention independent component analysis, nonnegative matrix factorization, sparse component analysis and/or smooth component analysis. However, there is considerably smaller number of methods for blind separation of pure components from recorded spectra assuming they are nonlinear mixtures of pure components. That number is reduced further when related nonlinear blind source separation problem is underdetermined, that is when number of pure components is greater than number of mixtures. One of the main reasons is that in an underdetermined scenario pure components are correlated (statistically dependent), that is their mass spectra overlap.

Subsequent paragraphs discuss state-of-the-art algorithms for nonlinear blind source separation problem. Patents and patent applications related to nonlinear blind source separation are also discussed. Thereby, there is no method that is related to blind separation of correlated (overlapping) pure components from smaller number of nonlinear mixtures of mass spectra. Yet, underdetermined scenario is relevant for description of mixtures mass spectra of samples acquired in food, health and environment related studies. For example 497 unique chemical components were quantified in extracts of Arabidopsis thaliana leaf tissue in reference: Jonsson P, Johansson A I, Gullberg J, Trygg J, Jiye A, Grung B, Marklund S, Sjöström M, Antti H, Moritz T. High-throughput data analysis for detecting and identifying differences between samples in GC/MS-based metabolomic analyses, Analytical Chemistry 2005; 77: 5635-5642. Analysis of human adult urinary metabolome by liquid chromatography-mass spectrometry revealed presence of 1484 components, while 384 of them were characterized by matching their spectra with references stored in libraries: Roux A, Xu Y, Heilier J-F, Olivier M-F, Ezan E, Tabet J-C, Junot C, “Annotation of the human adult urinary metabolome and metabolite identification using ultra high performance liquid chromatography coupled to a linear quadrupole ion trap-orbitrap mass spectrometer,” Analytical Chemistry 2012; 84: 6429-6437. Such large number of components present in much smaller number of mixtures will overlap, that is pure components will be correlated. Overlapping is also caused by chemical reason, that is when two or more pure components elute from chromatography column close to each other in time their peaks overlap partially or completely: Ni Y, Qiu Y, Jiang W, Suttlemyre K, Su M, Zhang W, Jia W, Du X, “ADAP-GC 2.0 Deconvolution of Coeluting Metabolites from GC/TOF-MS Data for Metabolomic Studies,” Analytical Chemistry 2012; 84: 6619-6629. Above discussion supports the statement that metabolic profiling, identification and quantification of small-molecule pure components present in biological samples is one of the most challenging tasks in chemical biology: Nicholson J K, Lindon J C, “Systems biology: Metabonomics,” Nature 2008; 455 (7216): 1054-1056.

In relation to nonlinear nonnegative underdetermined blind source separation problem composed of statistically dependent sources, that is the aim of the present invention, state-of-the-art algorithms for nonlinear blind source separation have at least one of the several deficiencies: (i) they assume that number of mixtures is equal to or greater than the unknown number of sources; (ii) they do not take into account nonnegativity constraint that is chemically justified when sources are pure components represented by mass spectra; (iii) they assume that source signals are statistically independent and, sometimes, individually correlated. None of these assumptions applies to the nonlinear underdetermined blind source separation problem that is the aim of the present invention.

Algorithms developed for nonlinear blind source separation problem that are cited below assume that number of sources is less than or equal to the number of mixtures. Thus, they are incapable to solve underdetermined blind source separation problem that is the aim of the present invention. These algorithms are published in: K. Zhang, and L. Chan, “Minimal Nonlinear Distortion Principle for Nonlinear Independent Component Analysis,” J Mach. Learn. Res., vol. 9, pp. 2455-2487, 2008; D. N. Levin, “Using state space differential geometry for nonlinear blind source separation,” J. Appl. Phys., vol. 103, 044906:1-12, 2008; D. N. Levin, “Performing Nonlinear Blind Source Separation With Signal Invariants,” IEEE Trans. Sig. Proc., vol. 58, pp. 2131-2140, 2010; A. Taleb, and C. Jutten, “Source Separation in Post-Nonlinear Mixtures,” IEEE Trans. Sig. Proc., vol. 47, pp. 2807-2820, 1999; L. T. Duarte, R. Suyama, R. Attux, J. M. T. Romano, C. Jutten, “Blind compensation of nonlinear distortions via sparsity recovery,” Proc. EUSIPCO 2012, pp. 2362-2366, Bucharest, Romania, Aug. 27-31, 2012; E. F. S. Filho, J. M. de Seixas, L. P. Calôba, “Modified post-nonlinear ICA model for online neural discrimination,” Neurocomputing, vol. 73, pp. 2820-2828, 2010; T. V. Nguyen, J. C. Patra, A. Das, “A post nonlinear geometric algorithm for independent component analysis,” Digital Signal Proc., vol. 15, pp. 276-294, 2005; A. Ziehe, M. Kawanabe, S. Harmeling, K. R. Miiller, “Blind Separation of Post-Nonlinear Mixtures Using Gaussianizing Transformations And Temporal Decorrelation,” J. Mach. Learn. Res., vol. 4, pp. 1319-1338, 2003; K. Zhang, L. W. Chan, “Extended Gaussianization Method for Blind Separation of Post-Nonlinear Mixtures,” Neural Computation, vol. 17, pp. 425-452, 2005.

Algorithms developed for nonlinear blind source separation problem that are cited below assume that source signals are statistically independent. Thus, they are incapable to solve underdetermined blind source separation problem that is comprised of nonnegative statistically dependent sources and that is aim of the present invention. These algorithms are published in: S. Harmeling, A. Ziehe, and M. Kawanabe, “Kernel-Based Nonlinear Blind Source Separation,” Neural Computation, vol. 15, pp. 1089-1124, 2003; D. Martinez, and A. Bray, “Nonlinear Blind Source Separation Using Kernels. IEEE Tr. on Neural Networks vol. 14, pp. 228-235, 2003; L. Almeida, “MISEP-Linear and nonlinear ICA based on mutual information,” J. Mach. Learn. Res., vol. 4, pp. 1297-1318, 2003; Z. L. Sun, D. S. Huang, C. H. Zheng, L. Shang, “Using batch algorithm for kernel blind source separation,” Neurocomputing, vol. 69, pp. 273-278, 2005.

Algorithm described in the paper: S. V. Vaerenbergh, I. Santamaria, “A Spectral Clustering Approach to Underdetermined Postnonlinear Blind Source Separation of Sparse Sources,” IEEE Trans. Neural Net., vol. 17, No. 3, pp. 811-814, 2006, is developed for nonlinear underdetermined blind source separation problem composed of nonnegative sources. The assumption made by the algorithm is that set of observation indexes exist such that each source component is present alone in at least one of these observation points. That assumption is too strong for the considered problem in mass spectrometry where number of pure components can be large and therefore they are expected to overlap significantly. That is especially the case if the resolution of the mass spectrometer is low. Thus, discussed algorithm is incapable to solve underdetermined blind source separation problem that is the aim of the present invention.

Algorithms cited below are developed for nonnegative matrix factorization (NMF) in reproducible kernel Hilbert space (RKHS) of functions. These algorithms are only applied to classification and regression problems in RKHS. That is, they were not tested on linear blind source separation problems in RKHS. In addition to that, employed NMF algorithms are based on nonnegativity constraints only. That, however, is not enough to separate pure components that are essentially unique. For that sparseness constraint is necessary. Thus, these algorithms are incapable to solve nonlinear underdetermined blind source separation problem that is the aim of the present invention. Discussed algorithms are published in: I. Buciu, N. Nikolaidis, and I. Pitas, “Nonnegative Matrix Factorization in Polynomial Feature Space,” IEEE Trans. on Neural Networks, vol. 19, pp. 1090-1100, 2007; D. Zhang, and W. Liu, “An Efficient Nonnegative Matrix Factorization Approach in Flexible Kernel Space,” Proc. of the 21st International Joint Conference on Artificial Intelligence (IJCAI'09), Pasadena, Calif., 2009; S. Zafeiriou, and M. Petrou, “Non-linear Non-negative Component Analysis,” IEEE Trans. on Image Processing, vol. 19, no. 4, pp. 1050-1066, 2010; B. Pan, J. Lai, and W. S. Chen, “Nonlinear nonnegative matrix factorization based on Mercer kernel construction,” Pattern Recognition, vol. 44, pp. 2800-2810, 2011.

In the U.S. Pat. No. 7,804,062 “Blind extraction of pure component mass spectra from overlapping mass spectrometric peaks” a method for blind extraction of pure components from their linear mixtures is presented. Thereby, mixtures correspond to mass spectra recorded at different elution times and separation vector corresponds with the pure component that has the minimum entropy. The algorithm proposed in U.S. Pat. No. 7,804,062 is incapable to solve nonlinear underdetermined blind source separation problem that is the aim of the present invention.

U.S. Pat. No. 8,165,373 “Method of and system for blind extraction of more pure components than mixtures in 1D and 2D NMR spectroscopy and mass spectrometry combining sparse component analysis and single component points” considers linear underdetermined blind source separation problem in NMR spectroscopy and mass spectrometry. Thus, it is incapable to solve nonlinear underdetermined blind source separation problem that is the aim of the present invention. The same statement applies for the methods published in two journal papers: I. Kopriva, I. Jerić, “Blind separation of analytes in nuclear magnetic resonance spectroscopy and mass spectrometry: sparseness-based robust multicomponent analysis,” Analytical Chemistry, vol. 82, pp. 1911-1920, 2010; I. Kopriva, I. Jerić, “Multi-component Analysis: Blind Extraction of Pure Components Mass Spectra using Sparse Component Analysis,” Journal of Mass Spectrometry, vol. 44, issue 9, pp. 1378-1388, 2009.

Patent application CN 101972143 “Blind source extraction-based atrial fibrillation monitoring method” relates to blind source separation method for extraction of electrocardio signal for monitoring of atrial fibrillation disease. The blind source separation is performed after nonlinear dimensionality expansion to extract electrocardio signal. To achieve this wavelet transform is used together with spectral subtraction and prior information on heart rate parameters. Since the use of wavelet transform destroys nonnegativity proposed method is not applicable, even in the principle, to solve the underdetermined nonlinear blind source problem composed of statistically dependent pure components represented by mass spectra and that is the aim of the present invention.

Patent application WO 2008/076680 “Method and apparatus for using state space differential geometry to perform nonlinear blind source separation,” has also been published in two journal papers: D. N. Levin, “Using state space differential geometry for nonlinear blind source separation,” J. Appl. Phys., vol. 103, 044906:1-12, 2008; D. N. Levin, “Performing Nonlinear Blind Source Separation With Signal Invariants,” IEEE Trans. Sig. Proc., vol. 58, pp. 2131-2140, 2010. Proposed method is capable to separate source signals from nonlinear mixtures provided that they are separable in phase space. It means that joint probability density function of the source signals and their derivatives is product of marginal probability density functions in the same space. This implies that source signals are statistically independent in the phase space. There are two limitations of the proposed method in relation to the underdetermined nonlinear blind source problem composed of statistically dependent pure components represented by mass spectra and that is the aim of the present invention: (i) statistical independence assumption is not true for overlapped nonnegative pure components in mass spectrometry; (ii) the method proposed in WO 2008/076680 requires that number of mixtures be equal to or greater than the number of pure component signals. Thus, it cannot solve underdetermined blind source separation problem.

Algorithm described in the paper: I. Kopriva, I. Jerić, L. Brklja\check{c}ić, “Nonlinear mixture-wise expansion approach to underdetermined blind separation of nonnegative dependent sources,” Journal of Chemometrics, vol. 27, pp. 189-197, 2013, is developed for linear underdetermined blind separation of nonnegative dependent sources, whereas sources represented pure components mass spectra and were required to have amplitudes that are sparsely distributed. This algorithm is in part related to the aim of the present invention: the method for nonlinear underdetermined blind separation of nonnegative dependent sources. Even though nonlinear blind source separation problem is more difficult to solve than its linear counterpart it is unexpectedly found that solution of both problems can be obtained under the same constraints imposed on nonnegative dependent sources: they have to be sparse in support and amplitude and pure components in mass spectrometry satisfy these constraints. However, as opposed to the linear blind source separation problem, errors introduced in nonlinear blind source separation problem by nonlinear mixing process have to be reduced further.

In the present invention errors introduced by nonlinear mixing process are reduced by a combination of methods that preprocess recorded matrix of mixtures mass spectra: (i) robust principal component analysis (RPCA): E. J. Candès, X. Li, Y. Ma and J. Wright, “Robust Principal Component Analysis?”, Journal of the ACM, vol. 58, No. 3, Article 11, May, 2011; V. Chandrasekaran, S. Sanghavi, P. A. Paririlo, A. S. Wilsky, “Rank-Sparsity Incoherence for Matrix Decomposition,” SIAM Journal on Optimization, vol. 21, No. 2, pp. 572-596, 2011.

RPCA decomposes corrupted recorded matrix of mixture mass spectra into low-rank matrix and sparse error matrix. The corruption is due to nonlinear mixing process that introduces error terms in the Taylor expansion of recorded matrix of mixture mass spectra. Thereby, low-rank matrix contains mixtures mass spectra with reduced error terms; (ii) hard thresholding of the recorded matrix of mixture mass spectra: Donoho D. L. De-Noising by Soft-Thresholding. IEEE Trans. Inf. Theory 1995; 41 (3): 613-627. Hard thresholding is a process that sets to zero coefficients of some vector or matrix if their absolute value is lower than the threshold; (iii) soft thresholding of the recorded matrix of mixture mass spectra: Donoho D. L. De-Noising by Soft-Thresholding. IEEE Trans. Inf. Theory 1995; 41 (3): 613-627. Soft thresholding is an extension of hard thresholding. First, it sets to zero elements of vector or matrix if their absolute values are lower than a threshold; (iv) trimmed thresholding of the recorded matrix of mixture mass spectra: H. T. Fang, D. S. Huang, “Wavelet de-noising by means of trimmed thresholding,” Proceedings of the 5th World Congress on Intelligent Control and Automation, Jun. 15-19, 2004, Hangzhou, P. R. China, pp. 1621-1624. Trimmed thresholding is between hard and soft thresholding. By selecting value of the proper parameter it can act as a hard thresholding, soft thresholding or in between.

Accordingly, it is the aim of the present invention to provide a method and system for blind separation of nonnegative dependent (overlapping) pure components from smaller number of nonlinear mixtures of mass spectra. Thereby, the pure components are identified by correlating separated components with the components stored in the library of referenced compounds.

### SUMMARY OF THE INVENTION

This aim is achieved by a method for blind separation of nonnegative correlated pure components mass spectra from smaller number of nonlinear mixture mass spectra by means of empirical kernel map-based mappings of pre-processed matrix of mixture mass spectra and sparseness and nonnegativity constrained factorization of mapped pre-processed matrices, characterised in that said nonlinear underdetermined nonnegative blind separation of correlated sources comprises the following steps:
recording and storing the mixtures data X, where X is nonnegative data matrix comprised of N≧2 rows that correspond to mixture mass spectra and R columns that correspond to observations at different mass-to-charge (m/z) ratios;
scaling the mixture data matrix by maximal element of X, xₘₐₓ:

X=X/xₘₐₓ [I]
that yields new data matrix X such that 0≦xₙᵣ≦1, n=1, . . . , N, and r=1, . . . , R,
representing scaled mixture data matrix in [I] by nonlinear mixture model:

X=f(S) [II]
where S stands for an unknown nonnegative matrix comprised of M>N rows {sₘ}ₘ₌₁^{M} that correspond with pure components mass spectra and R columns that correspond with intensities at different m/z ratios; f(S) implies that nonlinear mapping is performed column-wise: xᵣ=f(sᵣ) r=1, . . . , R, whereas f(sᵣ)=[ƒ₁(sᵣ) . . . ƒ_{N}(sᵣ)]^{T} and {ƒₙ: R₀₊^{M}→R₀₊}ₙ₋₁^{B}. Scaling [I] implies that 0≦sₘᵣ≦1, m=1, . . . , M and r=1, . . . , R,
using mixed state probabilistic model for the amplitudes of the pure components mass spectra sₘᵣ:

p(sᵣ)=ρₘδ(sₘᵣ)+(1−ρₘ)δ*(sₘᵣ)ƒ(sₘᵣ) [III]
where δ(sₘᵣ) is an indicator function and δ*(sₘₜ)=1−(sₘᵣ) is its complementary function, ρₘ stands for probability that sₘᵣ is equal to zero. Thus, 1−ρₘ stands for probability that sₘᵣ is greater than zero. ƒ(sₘᵣ) is continuous probability density function that models sparse probability distribution of the amplitude sₘᵣ.
representing [II] by using truncated Taylor expansion:

$$X = {G}_{s} S + {G}_{{s}^{2}} [ {\{ {s}_{m 1} {s}_{{m}_{2}} \}}_{{m}_{1} , {m}_{2} = 1}^{M} ] + HOT \qquad [ IV ]$$

where {sₘ_{₁}sₘ_{₂}}ₘ_{₁}ₘ_{₂}₌₁^{M} stand for second order monomials that are cross-products between pure components {sₘ}ₘ₌₁^{M}, Gₛ and Gₛ_{²} are matrices of appropriate dimensions and HOT stands for higher-order terms that include monomials of order greater than 2,
apply robust principal component analysis to X in [IV] to obtain:

X=A+E [V]
where

$$A \approx {G}_{s} S + {G}_{{s}^{2}} [ {\{ {s}_{{m}_{1}} {s}_{{m}_{2}} \}}_{{m}_{1} , {m}_{2} = 1}^{M} ]$$

stands for low-rank matrix composed of linear combination of original pure components and linear combination of second order monomials that represent new components correlated with the original ones, and E≈HOT stands for sparse matrix that represents error terms associated with higher-order monomials,
applying hard threshodling operator to X in [IV] to obtain:

$$B \approx {G}_{s} S + {G}_{{s}^{2}} [ {\{ {s}_{{m}_{1}} {s}_{{m}_{2}} \}}_{{m}_{1} , {m}_{2} = 1}^{M} ] \qquad [ VI ]$$

where B stands for hard thresholded version of X in [IV],
applying soft thresholding operator to X in [IV] to obtain:

$$C \approx {G}_{s} S + {G}_{{s}^{2}} [ {\{ {s}_{{m}_{1}} {s}_{{m}_{2}} \}}_{{m}_{1} , {m}_{2} = 1}^{M} ] \qquad [ VII ]$$

where C stands for soft thresholded version of X in [IV],
applying trimmed thresholding operator to X in [IV] to obtain:

$$D \approx {G}_{s} S + {G}_{{s}^{2}} [ {\{ {s}_{{m}_{1}} {s}_{{m}_{2}} \}}_{{m}_{1} , {m}_{2} = 1}^{M} ] \qquad [ VIII ]$$

where D stands for trimmed thresholded version of X in [IV],
using empirical kernel map for nonlinear mapping of A in [V] onto reproducible kernel Hilbert space:

$$Ψ ( A ) = [ \begin{matrix}κ ( {a}_{1} , {v}_{1} ) & … & κ ( {a}_{R} , {v}_{1} ) \\ … & … & … \\ κ ( {a}_{1} , {v}_{D} ) & … & κ ( {a}_{R} , {v}_{D} )\end{matrix} ] \qquad [ IX ]$$

where κ(aᵣ,v_{d}), r=1, . . . , R and d=1, . . . , D stands for positive symmetric kernel function and v_{d}, d=1, . . . , D stand for basis vectors that approximately span the same space as the low-rank mixing vectors: aᵣ, r=1, . . . , R.
using empirical kernel map for nonlinear mapping of B in [VI] onto reproducible kernel Hilbert space:

$$Ψ ( B ) = [ \begin{matrix}κ ( {b}_{1} , {v}_{1} ) & … & κ ( {b}_{R} , {v}_{1} ) \\ … & … & … \\ κ ( {b}_{1} , {v}_{D} ) & … & κ ( {b}_{R} , {v}_{D} )\end{matrix} ] \qquad [ X ]$$

where interpretation of Ψ(B) is equivalent to those of Ψ(A) in [IX],
using empirical kernel map for nonlinear mapping of C in [VII] onto reproducible kernel Hilbert space:

$$Ψ ( C ) = [ \begin{matrix}κ ( {c}_{1} , {v}_{1} ) & … & κ ( {c}_{R} , {v}_{1} ) \\ … & … & … \\ κ ( {c}_{1} , {v}_{D} ) & … & κ ( {c}_{R} , {v}_{D} )\end{matrix} ] \qquad [ XI ]$$

where interpretation of Ψ(C) is equivalent to those of Ψ(A) in [IX],
using empirical kernel map for nonlinear mapping of D in [VIII] onto reproducible kernel Hilbert space:

$$Ψ ( D ) = [ \begin{matrix}κ ( {d}_{1} , {v}_{1} ) & … & κ ( {d}_{R} , {v}_{1} ) \\ … & … & … \\ κ ( {d}_{1} , {v}_{D} ) & … & κ ( {d}_{R} , {v}_{D} )\end{matrix} ] \qquad [ XII ]$$

where interpretation of Ψ(D) is equivalent to those of Ψ(A) in [IX],
applying sparseness and nonnegativity constrained matrix factorization (sNMF) algorithms to [IX], [X], [XI] and [XII] to obtain estimates of the pure components {sₘ}ₘ₌₁^{M} and some of their cross-products {sₘ_{₁}sₘ_{₂}}ₘ_{₁}_{,m}_{₁}₌₁^{M}:

{ s̅ₘ^{A}}ₘ₌₁^{\hat{M}}=sNMF(Ψ(A)) [XIII]

{ s̅ₘ^{B}}ₘ₌₁^{\hat{M}}=sNMF(Ψ(B)) [XIV]

{ s̅ₘ^{C}}ₘ₌₁^{\hat{M}}=sNMF(Ψ(C)) [XV]

{ s̅ₘ^{D}}ₘ₌₁^{\hat{M}}=sNMF(Ψ(D)) [XVI]
where \hat{M} denotes overall number of components separated in [XIII], [XIV], [XV] and [XVI].
estimating further the pure components by correlating { s̅ₘ^{A}}ₘ₌₁^{\hat{M}} from [XIII], { s̅ₘ^{B}}ₘ₌₁^{\hat{M}} from [XIV], { s̅ₘ^{C}}ₘ₌₁^{\hat{M}} from [XV] and { s̅ₘ^{D}}ₘ₌₁^{\hat{M}} from [XVI], with the components stored in the library composed of J reference compounds {sⱼ^{ref}}ⱼ₌₁^{J}:

$$\begin{matrix}{c}_{mj}^{A} = {argmax}_{j = 1 , … , J} \frac{〈 {\overline{s}}_{m}^{A} , {s}_{j}^{ref} 〉}{‖ {\overline{s}}_{m}^{A} ‖ ‖ {s}_{j}^{ref} ‖} m = 1 , … , \hat{M} , & [ XVII ] \\ {c}_{mj}^{B} = {argmax}_{j = 1 , … , J} \frac{〈 {\overline{s}}_{m}^{B} , {s}_{j}^{ref} 〉}{‖ {\overline{s}}_{m}^{B} ‖ ‖ {s}_{j}^{ref} ‖} m = 1 , … , \hat{M} , & [ XVIII ] \\ {c}_{mj}^{C} = {argmax}_{j = 1 , … , J} \frac{〈 {\overline{s}}_{m}^{C} , {s}_{j}^{ref} 〉}{‖ {\overline{s}}_{m}^{C} ‖ ‖ {s}_{j}^{ref} ‖} m = 1 , … , \hat{M} , & [ XIX ] \\ {c}_{mj}^{D} = {argmax}_{j = 1 , … , J} \frac{〈 {\overline{s}}_{m}^{D} , {s}_{j}^{ref} 〉}{‖ {\overline{s}}_{m}^{D} ‖ ‖ {s}_{j}^{ref} ‖} m = 1 , … , \hat{M} , & [ XX ]\end{matrix}$$

where [symbol] s̅ₘ,sⱼ^{ref} [symbol] , [symbol] s̅ₘ^{B},sⱼ^{ref} [symbol] , [symbol] s̅ₘ^{C},sⱼ^{ref} [symbol] and [symbol] s̅ₘ^{D},sⱼ^{ref} [symbol] denote the inner products respectively between s̅ₘ^{A}, s̅ₘ^{B}, s̅ₘ^{C}, s̅ₘ^{D} and sⱼ^{ref}. ∥ s̅ₘ^{A}∥, ∥ s̅ₘ^{B}∥, ∥ s̅ₘ^{C}∥, ∥ s̅ₘ^{D}∥ and ∥sⱼ^{ref}∥ denote, respectively, l₂-norm of s̅ₘ^{A}, s̅ₘ^{B}, s̅ₘ^{C}, s̅ₘ^{D} and sⱼ^{ref}.
assign to each component in the library {sⱼ^{ref}}ⱼ₌₁^{J} components separated from [XIII], [XIV], [XV] and [XVI] that are indexed according to:

$$\begin{matrix}[ {c}^{A} , {m}_{A}^{*} ] = {argmax}_{m} {\{ {c}_{mj}^{A} \}}_{m = 1}^{{A}_{j}} & [ XXI ] \\ [ {c}^{B} , {m}_{B}^{*} ] = {argmax}_{m} {\{ {c}_{mj}^{B} \}}_{m = 1}^{{B}_{j}} & [ XXII ] \\ [ {c}^{C} , {m}_{C}^{*} ] = {argmax}_{m} {\{ {c}_{mj}^{C} \}}_{m = 1}^{{C}_{j}} & [ XXIII ] \\ [ {c}^{D} , {m}_{D}^{*} ] = {argmax}_{m} {\{ {c}_{mj}^{D} \}}_{m = 1}^{{D}_{j}} & [ XXIV ]\end{matrix}$$

where Aⱼ, Bⱼ, Cⱼ and Dⱼ respectively stand for number of separated components { s̅ₘ^{A}}ₘ₌₁^{\hat{M}}, { s̅ₘ^{B}}ₘ₌₁^{\hat{M}}, { s̅ₘ^{C}}ₘ₌₁^{\hat{M}} and { s̅ₘ^{D}}ₘ₌₁^{\hat{M}} associated respectively in [XVII], [XVIII], [XIX] and [XX] to reference component sⱼ^{ref}.
obtain final estimates of the candidates for pure components {ŝⱼ}ⱼ₌₁^{J} according:

$$I = {argmax}_{A , B , C , D} ( {c}^{A} , {c}^{B} , {c}^{C} , {c}^{D} ) \\ {\hat{s}}_{j} = {\overline{s}}_{{m}_{I}^{*}} j = 1 , … , J and I ∈ \{ A , B , C , D \} . \qquad [ XXV ]$$

separated components { s̅ₘ^{A}}ₘ₌₁^{\hat{M}}, { s̅ₘ^{B}}ₘ₌₁^{\hat{M}}, { s̅ₘ^{C}}ₘ₌₁^{\hat{M}} and { s̅ₘ^{D}}ₘ₌₁^{\hat{M}} that are not assigned to the pure components from the library {ŝⱼ}ⱼ₌₁^{J}, can be considered as candidates for new pure components.
presenting estimated candidates of pure components and candidates for new pure components from [XXV].

Further, this aim is achieved by a system for blind separation of nonnegative dependent pure components from smaller number of nonlinear mixtures of mass spectra comprising: mass spectrometer (1) for recording mixtures data X, an input storing device/medium (2) for storing the mixture data X, a processor (3) wherein code is implemented or carried out for executing a method according to anyone of the claims 1 to 13 based on mixtures mass spectra X stored in/on the input storing device/medium (2), an output storing device or medium (4) for storing the result of the method carried out by the processor.

Preferably, probability density function ƒ(sₘᵣ) in [III] is exponential density ƒ(sₘᵣ)=(1/μₘ)exp(−sₘᵣ/μₘ) where μm stands for most expected value of the exponential distribution used to model probability distribution of the random variable sₘᵣ, that is amplitude of the pure component sₘᵣ.

Preferably, robust principal component analysis [V] is obtained as a solution of the optimization problem: minimize ∥A∥_{•}+λ∥E∥₁ subject to: A+E=X. Thereby,

$${‖ A ‖}_{*} = {\sum }_{i = 1}^{I \leq N} {σ}_{i}$$

denotes nuclear norm (sum of singular values) and I≦N is a rank of matrix A;

$${‖ E ‖}_{1} = {\sum }_{n = 1}^{N} {\sum }_{r = 1}^{R} {e}_{nr}$$

denotes l-norm of E and λ≈1\sqrt{R} is a regularization constant.

Preferably, hard thresholding operator in [VI] is applied to X entry-wise according to:

$${b}_{nr} = HT ( {x}_{nr} ) = \{ \begin{matrix}{x}_{nr} & if {x}_{nr} \geq {τ}_{1} \\ 0 & if {x}_{nr} < {τ}_{1}\end{matrix} ,$$

n=1, . . . , N and r=1, . . . , R and τ₁ε[10⁻⁶, 10⁻⁴] stands for a threshold.

Preferably, soft thresholding operator in [VII] is applied to X entry-wise according to: cₙᵣ=ST(xₙᵣ)=max(0,xₙᵣ−τ₂), n=1, . . . , N and r=1, . . . , R, and τ₂ε[10⁻⁶, 10⁻⁴] stands for a threshold.

Preferably, trimmed thresholding operator in [VIII] is applied to X entry-wise according to:

$${d}_{nr} = TT ( {x}_{nr} ) = \{ \begin{matrix}{x}_{nr} \frac{{x}_{nr}^{α} - {τ}_{3}^{α}}{{x}_{nr}^{α}} & if {x}_{nr} \geq {τ}_{3} \\ 0 & if {x}_{nr} < {τ}_{3}\end{matrix} ,$$

n=1, . . . , N and r=1, . . . , R, τ₃ε[10⁻⁶, 10⁻⁴] stands for a threshold and α is a trade-off parameter between hard and soft thresholding. When α=1 trimmed thresholding equals soft thresholding. When α→∞ trimmed thresholding is equivalent to hard thresholding. Conveniently, α=3.5.

Preferably, positive symmetric kernel functions in [IX], [X], [XI] and [XII] are selected as Gaussian kernels: κ(aᵣ,v_{d})=exp(−∥aᵣ−v_{d}∥²/σ²), κ(bᵣ,v_{d})=exp(−∥bᵣ−v_{d}∥²/σ²), κ(cᵣ,v_{d})=exp(−∥cᵣ−v_{d}∥²/σ²) and κ(dᵣ,v_{d})=exp(−∥dᵣ−v_{d∥}²/σ²). Conveniently, σ²=1.

Advantageously, dimension D of the basis {v_{d}}_{d=1}^{D} in [IX], [X], [XI] and [XII] is D=R. In this case each column vector of the matrices A, B, C and D is a basis vector, that is {(vᵣ=aᵣ}ᵣ₌₁^{R}, {vᵣ=bᵣ}ᵣ₌₁^{R}, {vᵣ=cᵣ}ᵣ₌₁^{R} and {vᵣ=dᵣ}ᵣ₌₁^{R}. In this case the basis spans the same vector space as the empirical set of the column vectors of A, B, C and D. When number of spectral lines R is large, that is the case with the high-resolution mass spectrometers, selection D=R can lead to computationally intractable problem. In this case some basis selection algorithm can be used yielding basis {v_{d}}_{d=1}^{D} that satisfies: D<<R and span{v_{d}}_{d=1}^{D}≈span{aᵣ}ᵣ₌₁^{R}, respectively span{bᵣ}ᵣ₌₁^{R}, span{cᵣ}ᵣ₌^{R} and span{dᵣ}ᵣ₌₁^{R}. Preferably, k-means data clustering algorithm can be used for basis selection by clustering R column vectors in D clusters. Thereby, cluster centroids represent basis vectors.

Preferably, sparseness and nonnegativity constrained matrix factorization (sNMF) algorithm in [XIII], [XIV], [XV] and [XVI] is nonnegative matrix underapproximation (NMU) algorithm: N. Gillis, F. Glineur, “Using underapproximations for sparse nonnegative matrix factorization,” Pattern Recognition, vol. 43, pp. 1676-1687, 2010. Thereby, assumed number of pure components \hat{M} in [XIII], [XIV], [XV] and [XVI] can, in the absence of a priori information, be set to dimension of induced reproducible kernel Hilbert space, that is: \hat{M}=D.

Alternatively, if number of pure components M and maximal number of overlapping components K are known a priori, sNMF algorithm in [XIII], [XIV], [XV] and [XVI] is nonnegative matrix factorization algorithm constrained with l₀-norm of S̅: R. Peharz, F. Pernkopf, “Sparse nonnegative matrix factorization with l⁰ constraints,” Neurocomputing, vol. 80, pp. 38-46, 2012. In this case we set for the assumed number of pure components in [XIII], [XIV], [XV] and [XVI]: \hat{M}≈2M+M (M−1)/2. Maximal number of overlapped components in [XIII], [XIV], [XV] and [XVI] is set as \hat{K}≈2K+K(K−1)/2.

According to a further special embodiment, a method is applied to the identification of compounds in chemical synthesis, food quality inspection or pollution inspection; that is environment protection.

Preferably, said method is applied to the identification of compounds obtained from natural sources (microorganisms, plants and animals), metabolites and biomarkers present in biological fluids (urine, blood plasma, cerebrospinal fluid, saliva, amniotic fluid, bile, tears, etc.) or tissue extracts.

Furthermore, the present invention provides a computer-readable medium having computer executable instructions stored thereon, which, when executed by computer will cause the computer to carry out a method of the present invention.

In a preferred embodiment of the system, the output storing device is a printer or plotter and the output storing medium is a memory based device that is readable by computer.

The novelty of proposed method for nonlinear underdetermined blind separation of correlated pure components mass spectra in relation to state-of-the-art in nonlinear blind source separation is that use of the sparseness constraint across support and amplitude of the pure components mass spectra in combination with preprocessing of recorded matrix of mixtures spectra by robust principal component analysis, trimmed thresholding, hard thresholding and soft thresholding and empirical kernel maps-based mapping of the preprocessed matrix of mixtures mass spectra enables reduction of errors introduced by nonlinear mixtures. Thus, effect of generating new mixtures by means of empirical kernel map-based mapping prevails over errors associated with higher-order terms that are induced by nonlinear mixtures. Therefore, nonnegativity and sparseness constrained factorization of mapped preprocessed matrices of mixture mass spectra will yield estimates of the pure components present in the nonlinear mixtures mass spectra.

### BRIEF DESCRIPTION OF THE DRAWINGS

A more detailed description of the invention will be given with references to the following figures, in which:

FIG. 1 schematically illustrated a block diagram of a device for nonlinear underdetermined blind separation of nonnegative dependent pure components mass spectra according to the embodiment of the present invention;

FIG. 2 schematically describes nonlinear chemical reaction of peptide bond formation;

FIGS. 3A to 3I show 9 chromatograms recorded during nonlinear chemical reaction related to peptide bond formation.

FIGS. 3J to 3R show mass spectra of 9 nonlinear mixtures obtained by full integration of chromatograms shown correspondingly on FIGS. 3A to 3I.

FIGS. 4A to 4Y show pure components mass spectra;

FIG. 4Z shows normalized correlation coefficients between pure components for normalized correlations greater than 0.1;

FIG. 5A shows probability ρₘ according to [III] that amplitude of the pure component m is zero and that is estimated for m=1, . . . , 25 pure components mass spectra;

FIG. 5B shows most expected (mean) value μₘ when in the mixed state probabilistic model of the pure components mass spectra [III] continuous distribution of the amplitude is substituted by exponential distribution: ƒ(sₘᵣ)=(1/μₘ)exp(−sₘᵣ/μₘ). μₘ is estimated for m=1, . . . , 25 pure components mass spectra;

FIG. 5C shows probability that amplitude of the pure components mass spectra occurs in some in the interval [0, A] where 0.01≦A≦1;

FIGS. 6A to 6Y show estimated histograms (stars) and exponential probability density functions (squares) calculated with the mean values obtained by fitting theoretical exponential distribution to estimated histograms;

FIG. 7A shows best results in terms of maximal correlation, minimal correlation, mean correlation, number of estimated components with correlations greater than or equal to 0.6 and number of separated components assigned incorrectly to pure components achieved by nonnegative matrix underapproximation algorithm (NMU) with assumed number of pure components \hat{M}=25; nonnegative matrix factorization algorithm constrained with l₀ quasi-norm (NMF_L0) with assumed number of pure components \hat{M}=25, and assumed maximal number of overlapped components \hat{K}=4; empirical kernel map (EKM) based mapping of matrix of mixture mass spectra and NMU based factorization of mapped matrix with assumed number of pure components \hat{M} equal to dimension of mapped space D that is further equal to number of spectral lines R=9901; EKM based mapping of matrices of mixture mass spectra preprocessed by robust principal component analysis (RPCA), hard, trimmed and soft thresholding (HT, ST and TT) NMU based factorization in mapped space with assumed number of pure components \hat{M} equal to dimension of mapped space D that is further equal to number of spectral lines R=9901. Thereby, according to [XXV], components separated from each mapped preprocessed matrix of mixtures are assigned to components from the library according to highest correlation criterion.

FIG. 7B shows highest correlation coefficient between pure component from the library and component separated from mapped matrices of mixtures mass spectra by RPCA, HT, TT or ST. In addition to the value of correlation coefficient method information on preprocessing method is also given.

FIGS. 8A to 8Y show mass spectra of the separated pure components assigned to the true pure components (library of reference components) according to the highest correlation criterion.

### DETAILED DESCRIPTION OF THE INVENTION

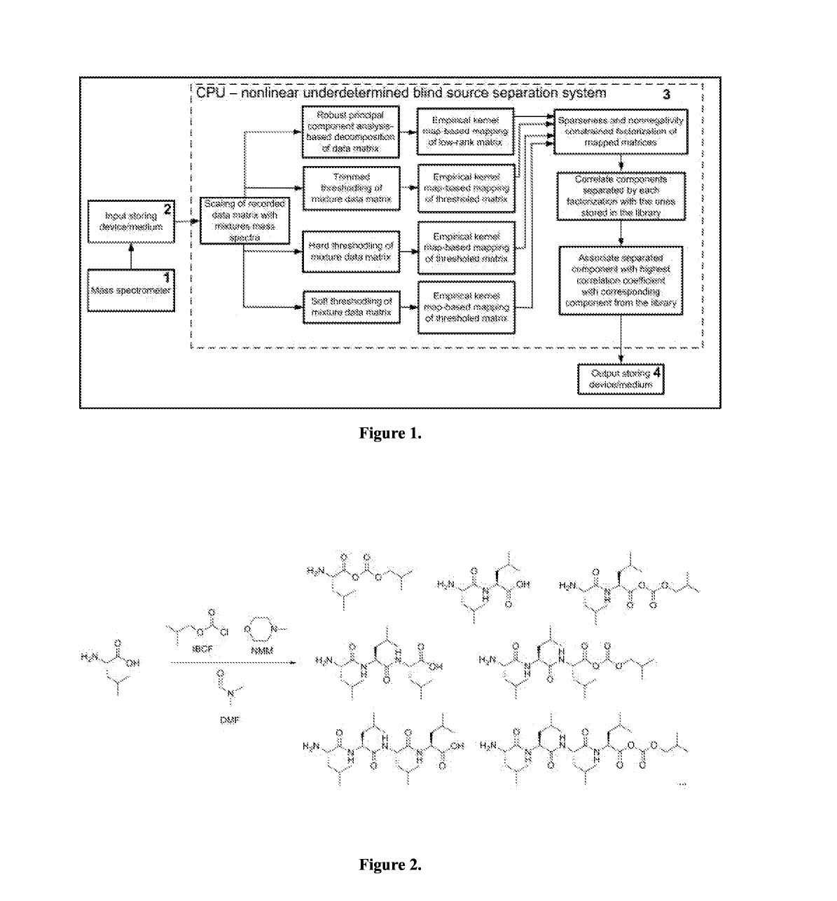

A schematic block-diagram of a device for blind separation of correlated pure components from smaller number of nonlinear mixtures mass spectra that is defined by equation [II] and employing methodology of robust principal component analysis, hard, trimmed and soft thresholding, empirical kernel map-based nonlinear mapping and nonnegativity and sparseness constrained matrix factorization according to an embodiment of the present invention is shown in FIG. 1. The device consists of: mass spectrometer 1 used to acquire nonlinear mixtures mass spectra; storing device 2 used to store acquired mass spectra; CPU 3 or computer where algorithm based robust principal component analysis, hard, trimmed and soft thresholding, empirical kernel map and nonnegativity and sparseness constrained factorization is implemented for nonlinear blind separation of nonnegative correlated pure components from smaller number of acquired nonlinear mixtures mass spectra; and output storing device 4 used to store and present separated pure components mass spectra.

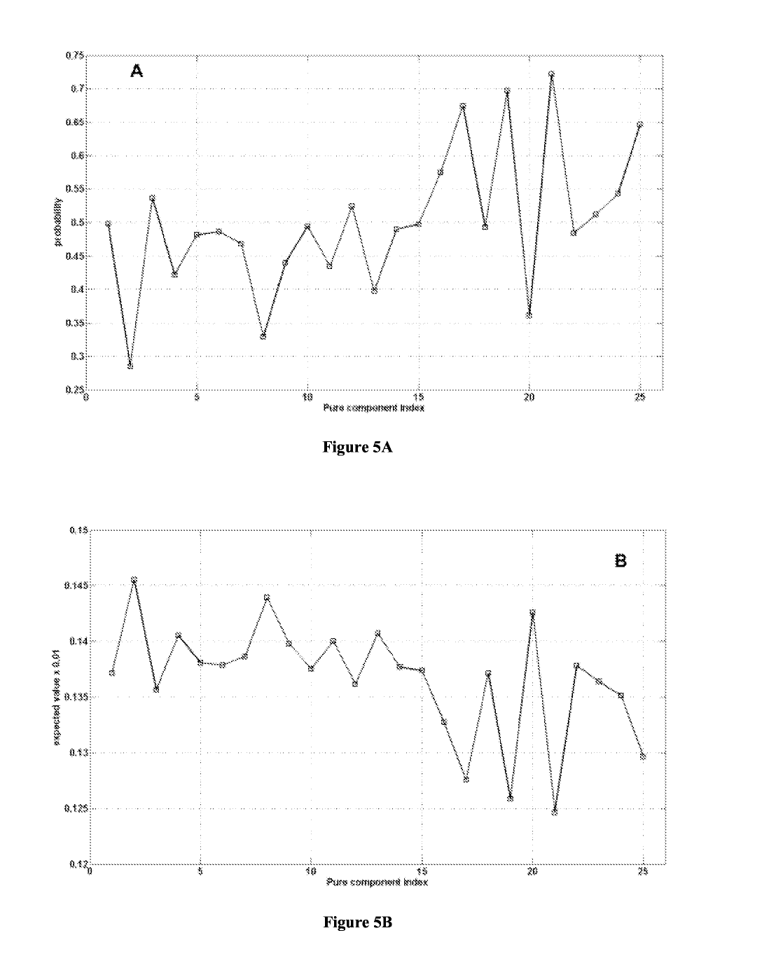

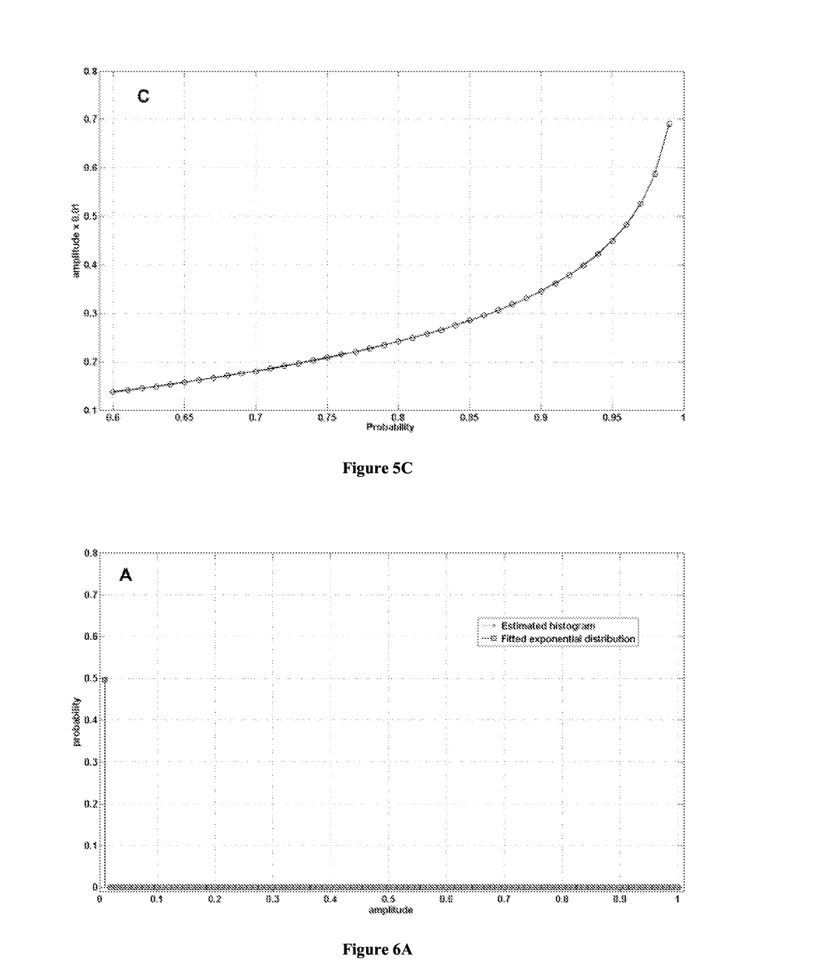

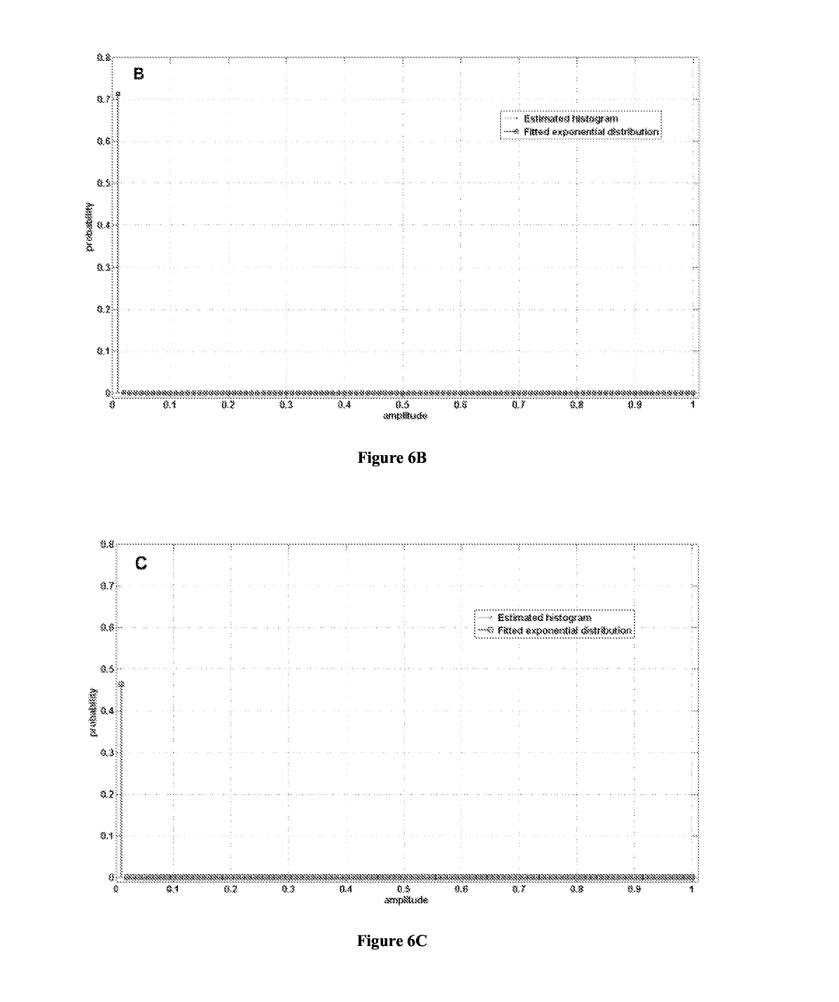

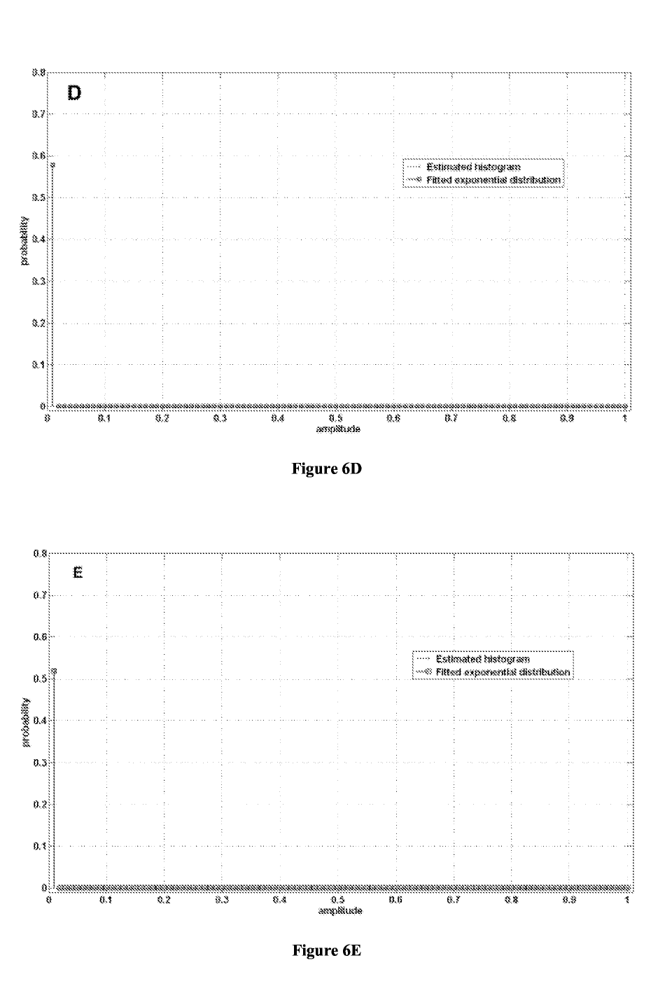

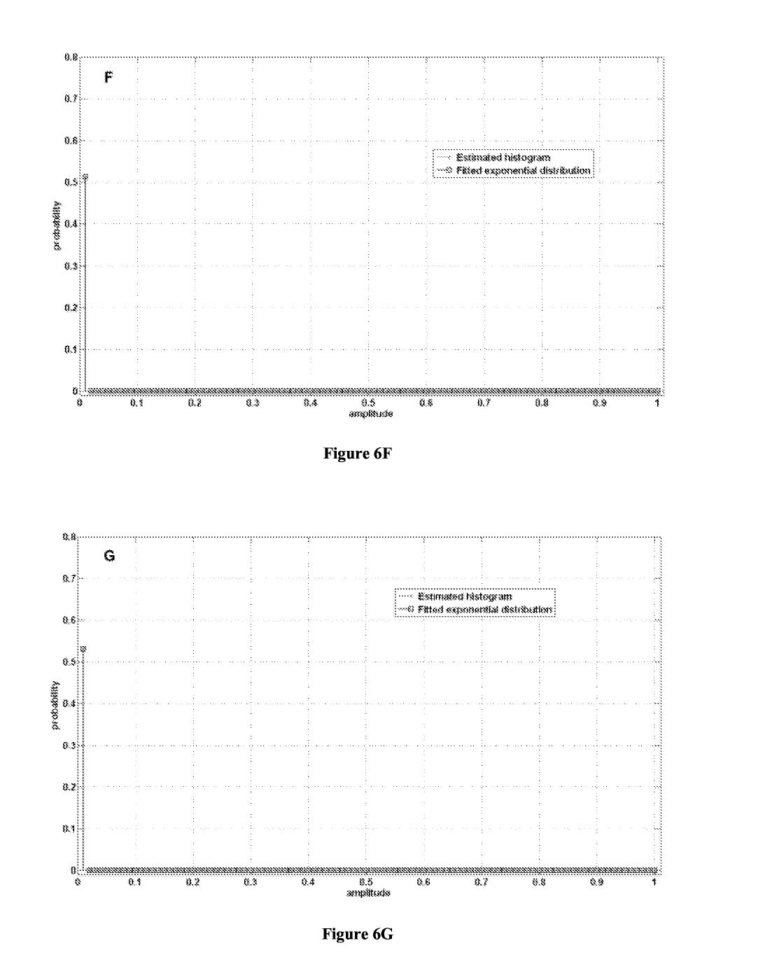

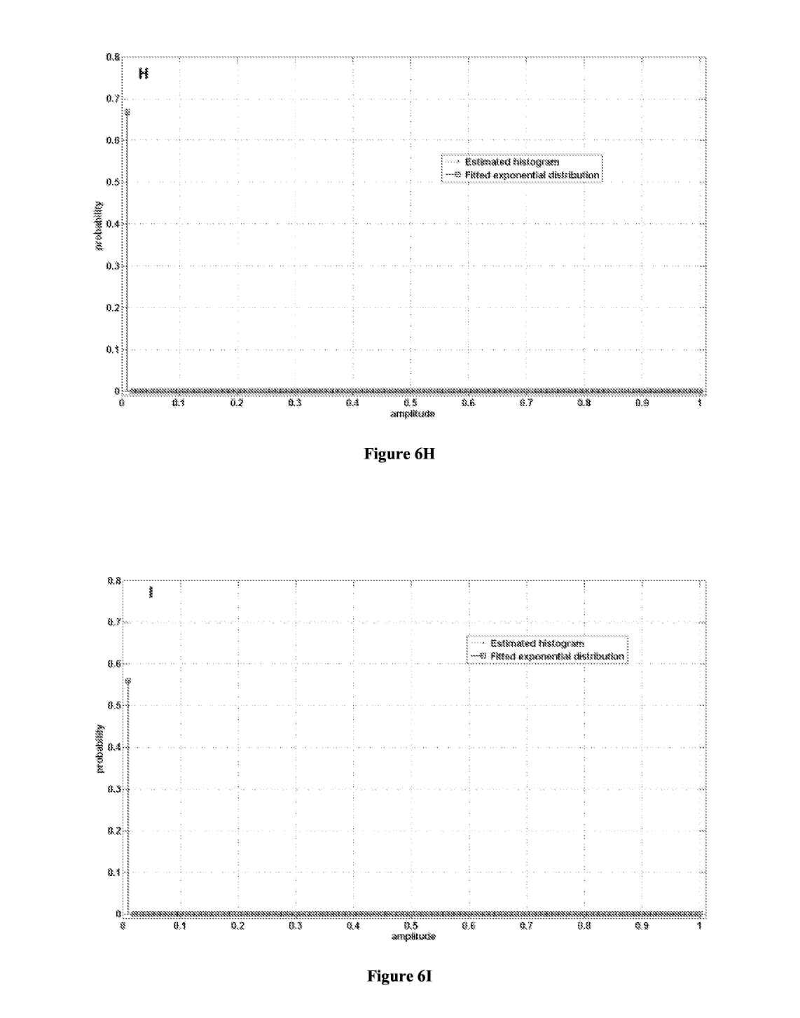

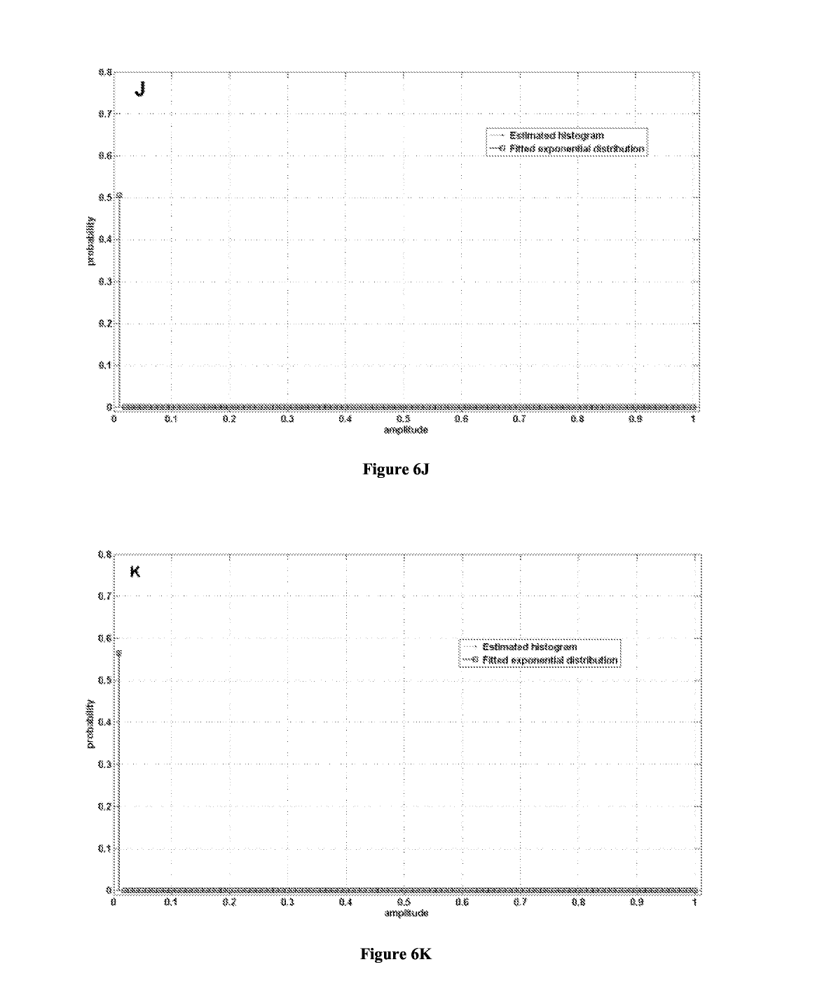

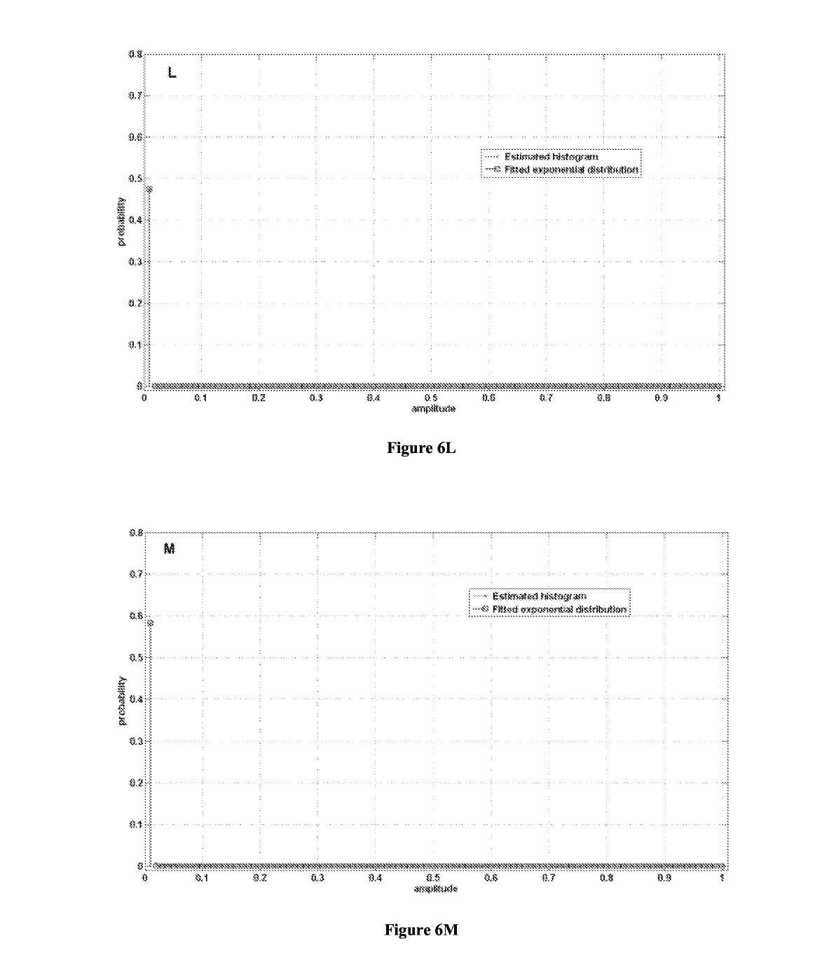

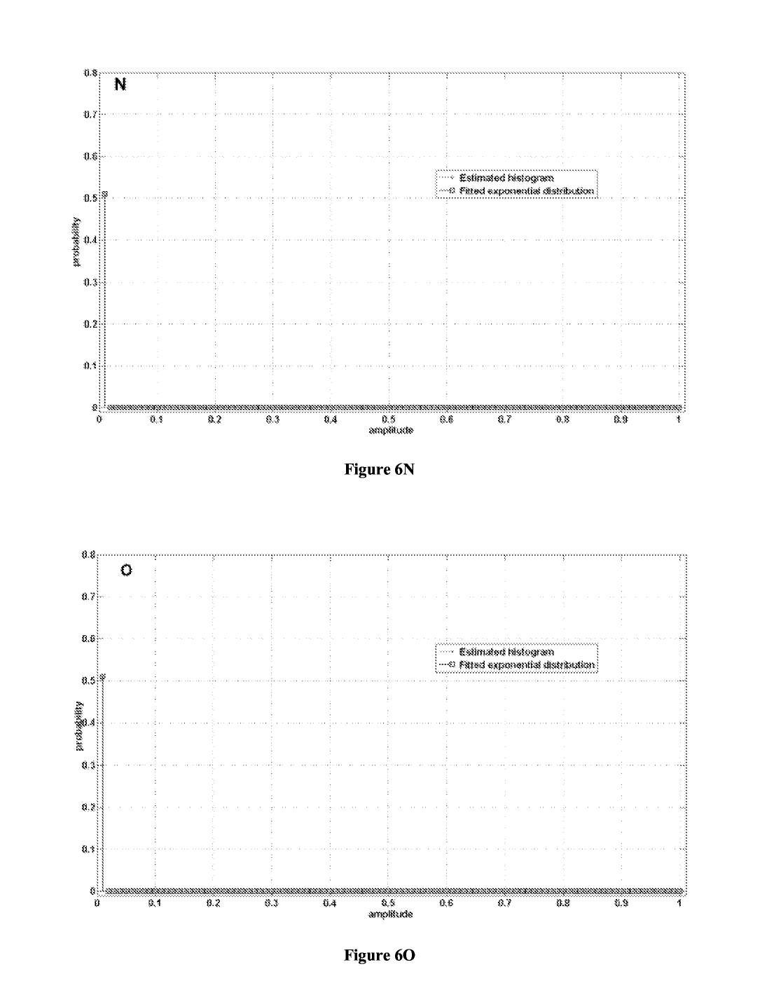

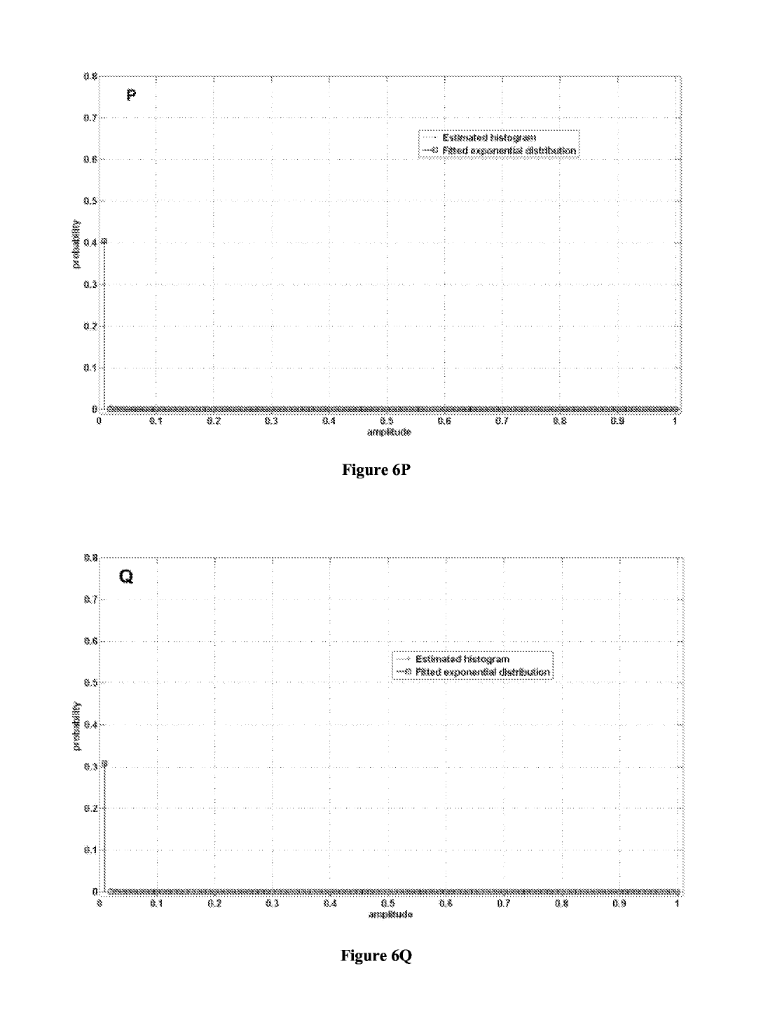

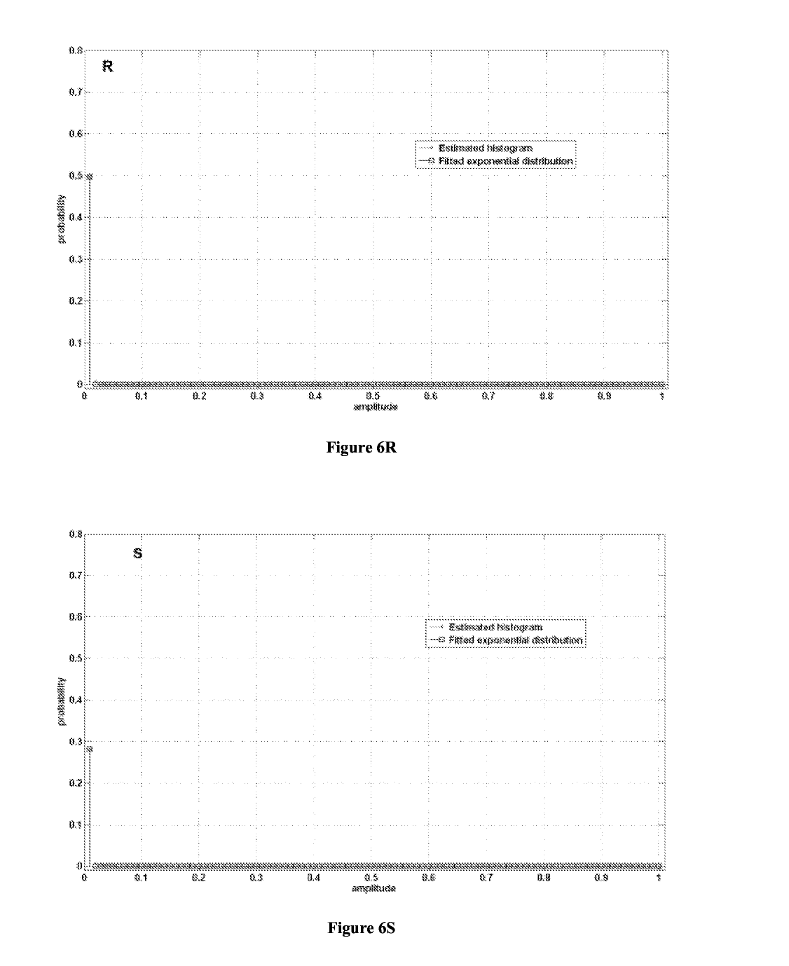

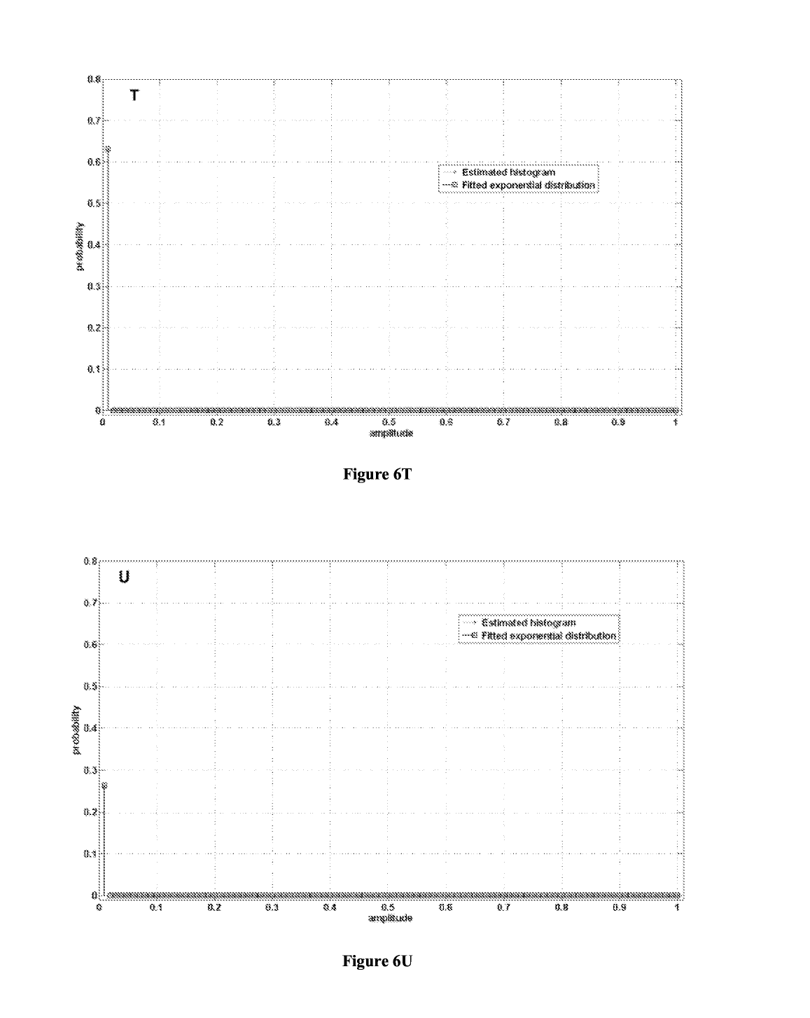

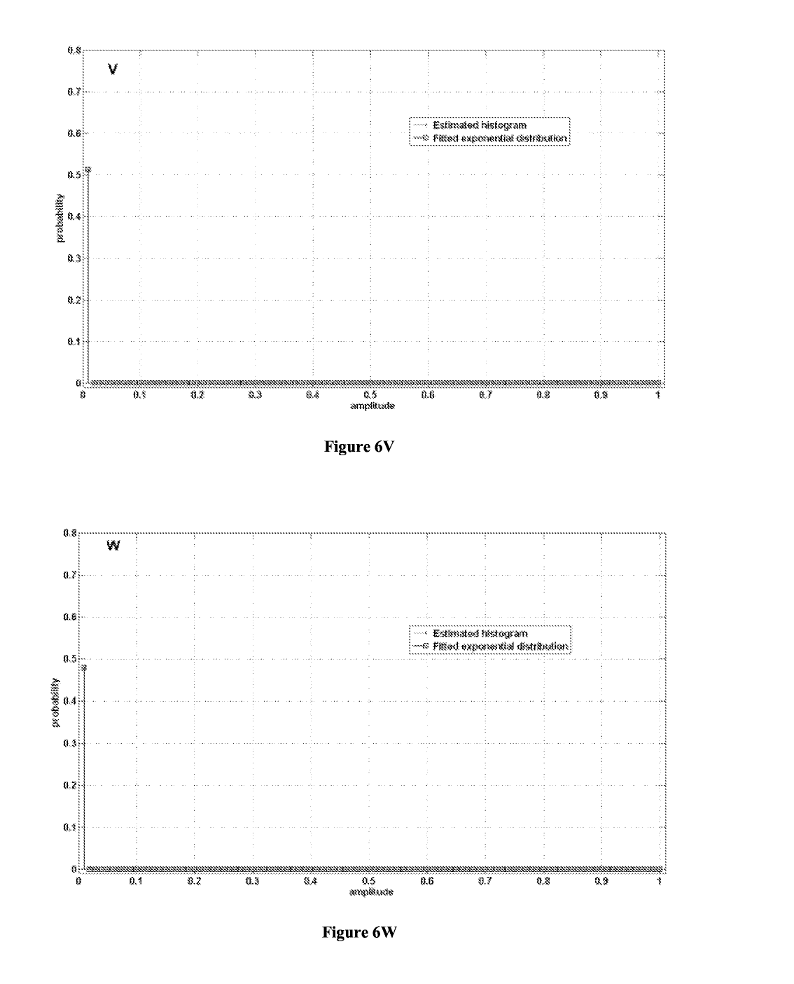

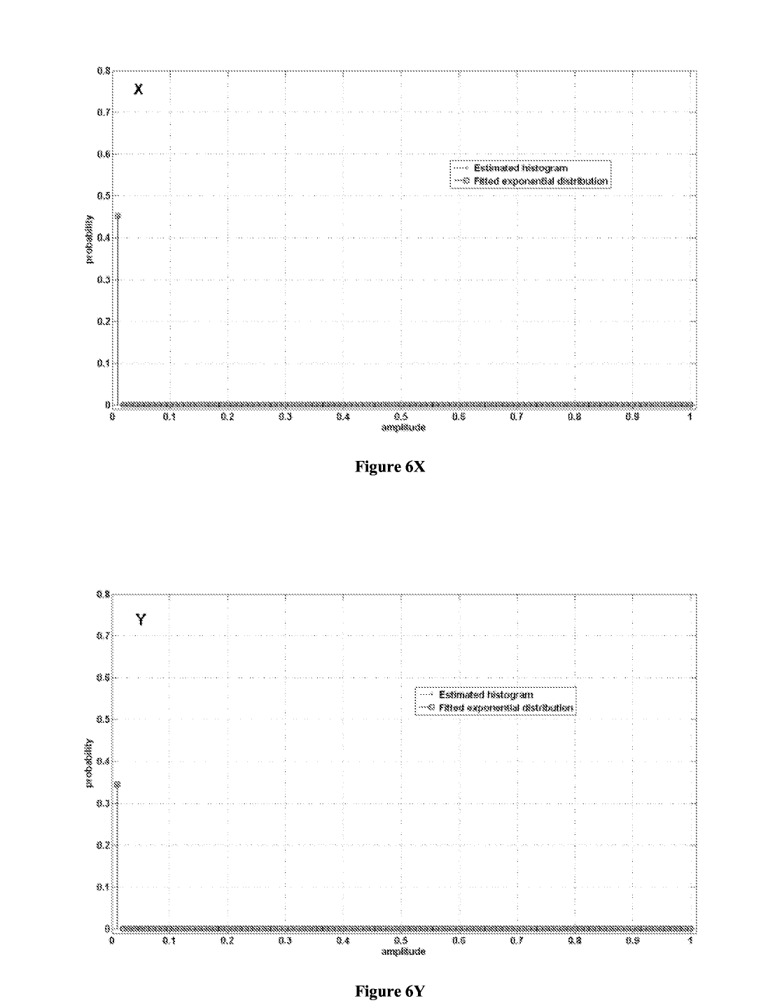

The procedure for processing acquired and stored mixtures mass spectra with the aim to blindly separate nonnegative correlated pure components from smaller number of nonlinear mixtures is implemented in the software or firmware in the CPU 3 and according to an embodiment of the present invention consists of the following steps: scaling of the acquired mixtures mass spectra according to equation [I] in order to constrain amplitudes of the mixture mass spectra to be in the range between 0 and 1; scaled mixture data are represented by nonlinear mixture model [II] which, due to mixed state sparse probabilistic model of the pure components mass spectra [III], can be expressed in truncated Taylor expansion [IV]. Taylor expansion [IV] ignores the cross-products of the pure components (also called monomials) of the order greater than 2. That is, due to the fact that 21 out of 25 pure components mass spectra are zero in 40% to 75% of the support (m/z ratios), see FIG. 5A. Thus, when more than 2 pure components are part of the monomial there is great probability that at least one of them will be zero and, therefore, the cross-product will be zero as well. Also, estimated mean values μₘ are in the interval μₘε[0.0012, 0.0015], see FIG. 5B. That is further supported by FIG. 5C which shows estimates of the probabilities that amplitude of the pure components mass spectra occurs in the [0, A] interval, where 0.01≦A≦1 is amplitude at particular m/z ratio. It can also be seen in FIGS. 6A to 6Y that theoretical exponential distribution calculated with estimated mean value approximates well empirical estimates in a form of histogram. All these implies that values of the monomials of the order greater than 2 are expected to be small; sparse distributions of the support and amplitude of the pure components mass spectra justifies: robust principal component analysis based decomposition of the mixture mass spectra [IV] in order to reduce the higher-order (error) terms that are induced by nonlinearities present in the mixtures and that yields [V], whereas A stands for low-rank matrix retained for further analysis and E stands for sparse error matrix; hard thresholding based preprocessing of the mixture mass spectra [IV] in order to reduce the higher-order (error) terms that are induced by nonlinearities present in the mixtures and that yields [VI], whereas B stands for error-reduced matrix retain for further analysis; soft thresholding based preprocessing of the mixture mass spectra [IV] in order to reduce the higher-order (error) terms that are induced by nonlinearities present in the mixtures and that yields [VII], whereas C stands for error-reduced matrix retain for further analysis; trimmed thresholding based preprocessing of the mixture mass spectra [IV] in order to reduce the higher-order (error) terms that are induced by nonlinearities present in the mixtures and that yields [VIII], whereas D stands for error-reduced matrix retain for further analysis. Empirical kernel map is used to map error-reduced matrices A, B, C and D into new matrices [IX], [X], [XI] and [XII], whereas number of mixtures D in [IX], [X], [XI] and [XII] is much greater than number of mixtures N in, respectively, [V], [VI], [VII] and [VIII]. Since mapping is nonlinear D mixtures in [IX], [X], [XI] and [XII] are linearly independent, that is they are not redundant. Thereby, it is of great importance that vector space spanned by the basis {v_{d}}_{d=1}^{D} approximates well the vector space respectively spanned by the empirical set of vectors {aᵣ}ᵣ₌₁^{R}, {bᵣ}ᵣ₌₁^{R}, {cᵣ}ᵣ₌₁^{R} and {dᵣ}ᵣ₌₁^{R}. To this end, setting D=R is the best choice because each mixing vector is the basis vector. That, however, can yield computationally intractable problem when resolution of the mass spectrometer is high and, consequently, R is large. In such a case it is possible to find a basis that spans low-dimensional subspace that approximates well the vector space spanned by the empirical set of vectors {aᵣ}ᵣ₌₁^{R}, {bᵣ}ᵣ₌₁^{R}, {cᵣ}ᵣ₌₁^{R} and {dᵣ}ᵣ₌₁^{R}. For this purpose data clustering algorithms or specialized basis selection algorithms can be used. Nonnegativity and sparseness constrained factorizations in [XIII], [XIV], [XV] and [XVI] yield estimates of the pure components as well as second order terms that represent new components correlated with the original pure components. Separated components from [XIII], [XIV], [XV] and [XVI] are correlated respectively in [XVII], [XVIII], [XIX] and [XX] with the reference pure components stored in the library. Separated components with the highest normalized correlation coefficient are according to [XXV] assigned to pure components and represent pure components candidates that are stored and presented in the output device or medium 4.

In detail, according to an embodiment of the present invention procedure for blind separation of nonnegative correlated pure components from smaller number of nonlinear mixtures mass spectra consists of the following steps:
recording two or more mixtures mass spectra X by means of mass spectrometer 1,
storing recorded mixture mass spectra on the input storing device or medium 2,
scaling stored mixture mass spectra according to equation [I] and representing them by nonlinear mixture model [II] as a nonlinear mapping f of the unknown matrix of pure components S,
based on mixed state sparse probabilistic model [III] of the pure components mass spectra representing mixtures mass spectra data matrix [II] with truncated Taylor expansion [IV],
applying robust principal component analysis on X in [IV] to reduce higher-order error terms due to nonlinear mixing process and that yields A in [V],
applying hard thresholding operator on X in [IV] to reduce higher-order error terms due to nonlinear mixing process and that yields B in [VI],
applying soft thresholding operator on X in [IV] to reduce higher-order error terms due to nonlinear mixing process and that yields C in [VII],
applying trimmed thresholding operator on X in [IV] to reduce higher-order error terms due to nonlinear mixing process and that yields D in [VIII],
using empirical kernel map for nonlinear mapping of A in [V] to obtain new mixtures data matrix Ψ(A) in [IX] with greater number of linearly independent mixtures than in the original mixtures data matrix X in [IV] and its approximation A in [V],
using empirical kernel map for nonlinear mapping of B in [VI] to obtain new mixtures data matrix Ψ(B) in [X] with greater number of linearly independent mixtures than in the original mixtures data matrix X in [IV] and its approximation B in [VI],
using empirical kernel map for nonlinear mapping of C in [VII] to obtain new mixtures data matrix Ψ(C) in [XI] with greater number of linearly independent mixtures than in the original mixtures data matrix X in [IV] and its approximation C in [VII],
using empirical kernel map for nonlinear mapping of D in [VIII] to obtain new mixtures data matrix Ψ(D) in [XII] with greater number of linearly independent mixtures than in the original mixtures data matrix X in [IV] and its approximation D in [VIII],
applying nonegativity and sparseness constrained matrix factorization algorithm to mapped matrix Ψ(A) in [IX] to separate pure components and their higher (second)-order monomials in [XIII],
applying nonegativity and sparseness constrained matrix factorization algorithm to mapped matrix Ψ(B) in [X] to separate pure components and their higher (second)-order monomials in [XIV],
applying nonegativity and sparseness constrained matrix factorization algorithm to mapped matrix Ψ(C) in [XI] to separate pure components and their higher (second)-order monomials in [XV],
applying nonegativity and sparseness constrained matrix factorization algorithm to mapped matrix Ψ(D) in [XII] to separate pure components and their higher (second)-order monomials in [XVI],
correlating separated components from [XIII], [XIV], [XV] and [XVI], with the components stored in the library composed of reference compounds,
assign to each component in the library components separated from [XIII], [XIII], [XIV] and [XV] that, respectively, are indexed according to [XXI], [XXII], [XXIII] and [XXIV],
obtaining final estimates of the candidates for pure components and candidates for new pure components according to [XXV],
storing and presenting estimated candidates of pure components and candidates for new pure components.

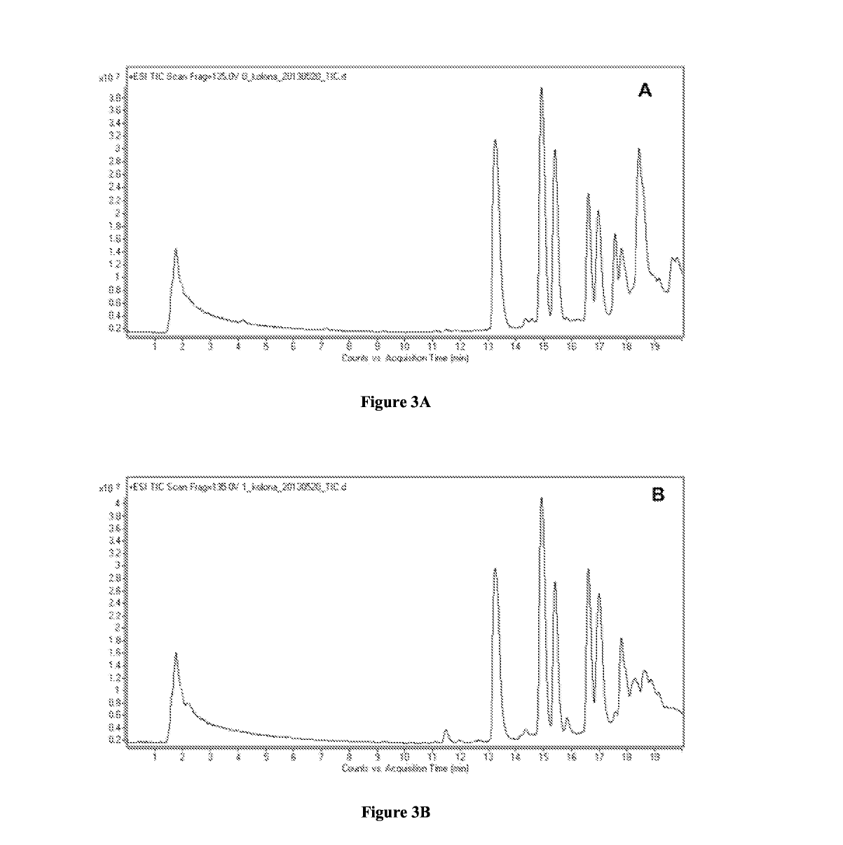

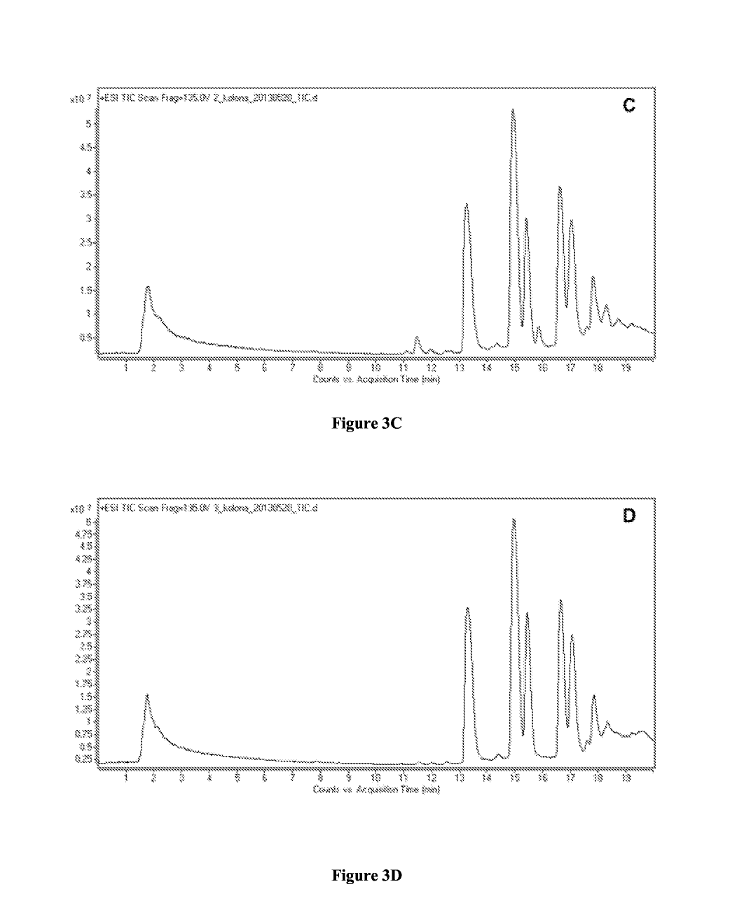

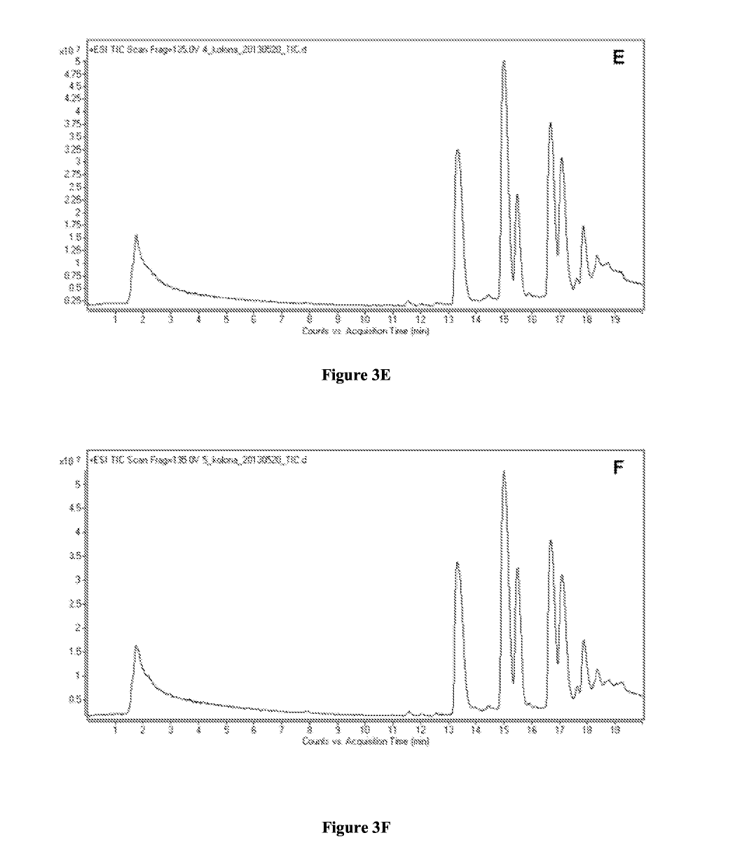

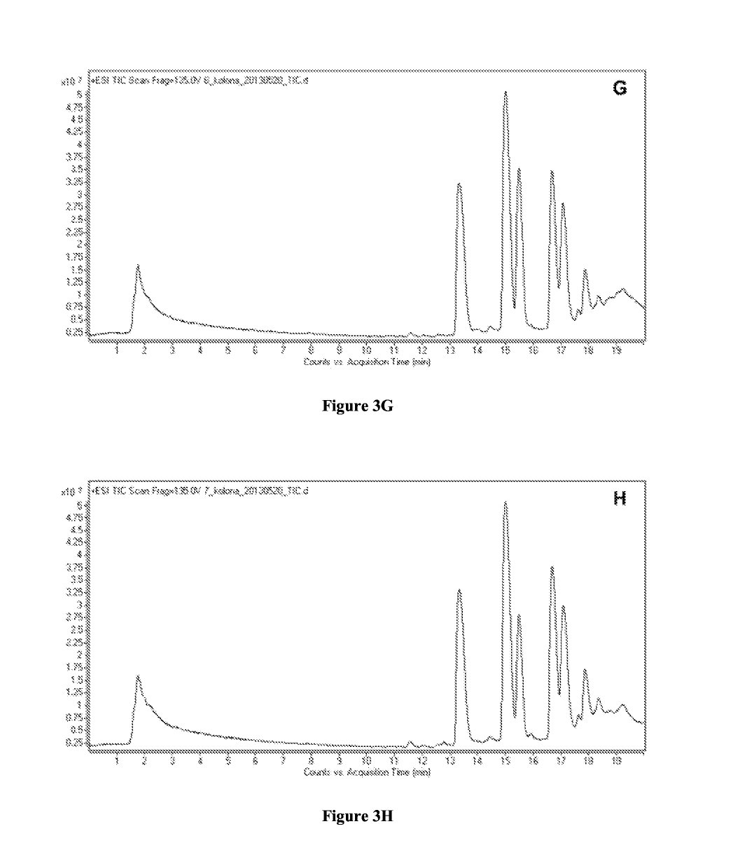

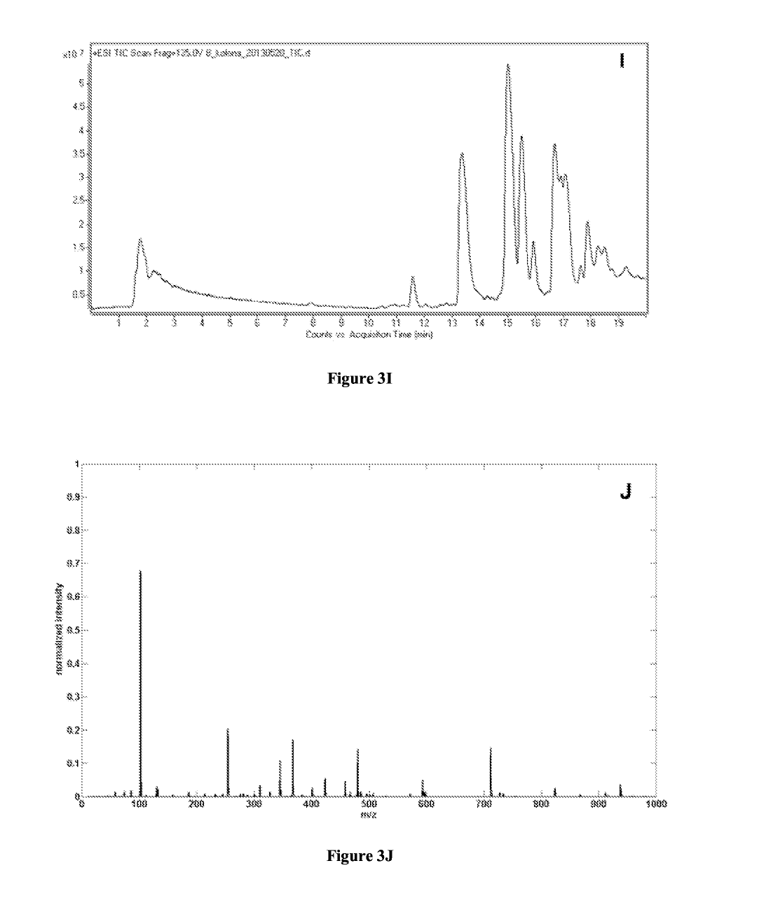

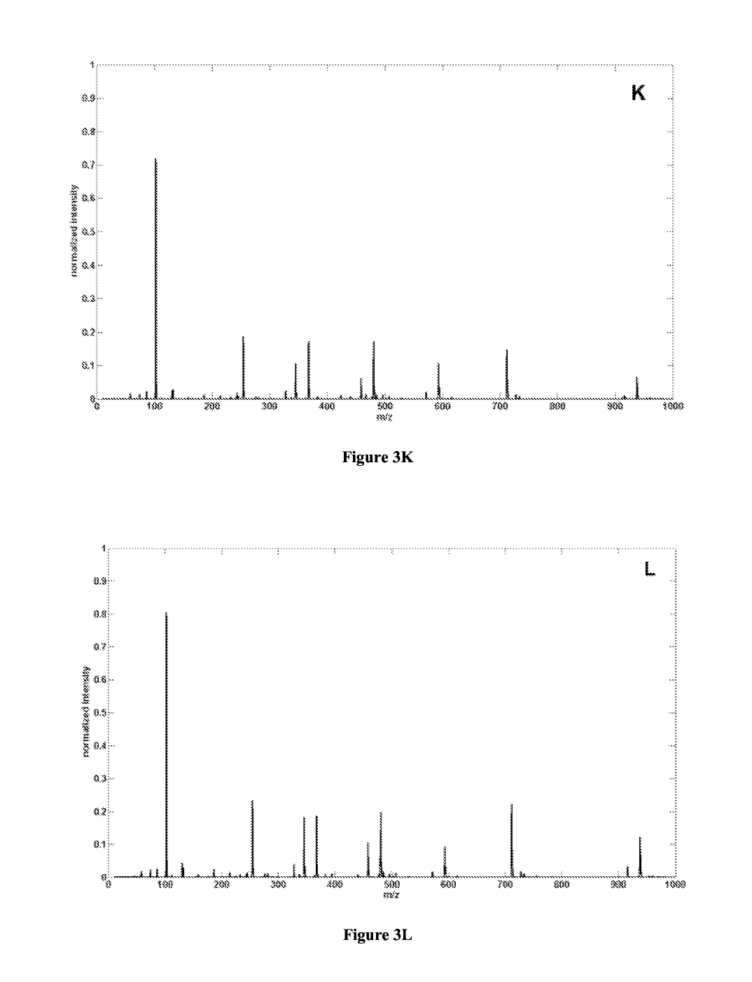

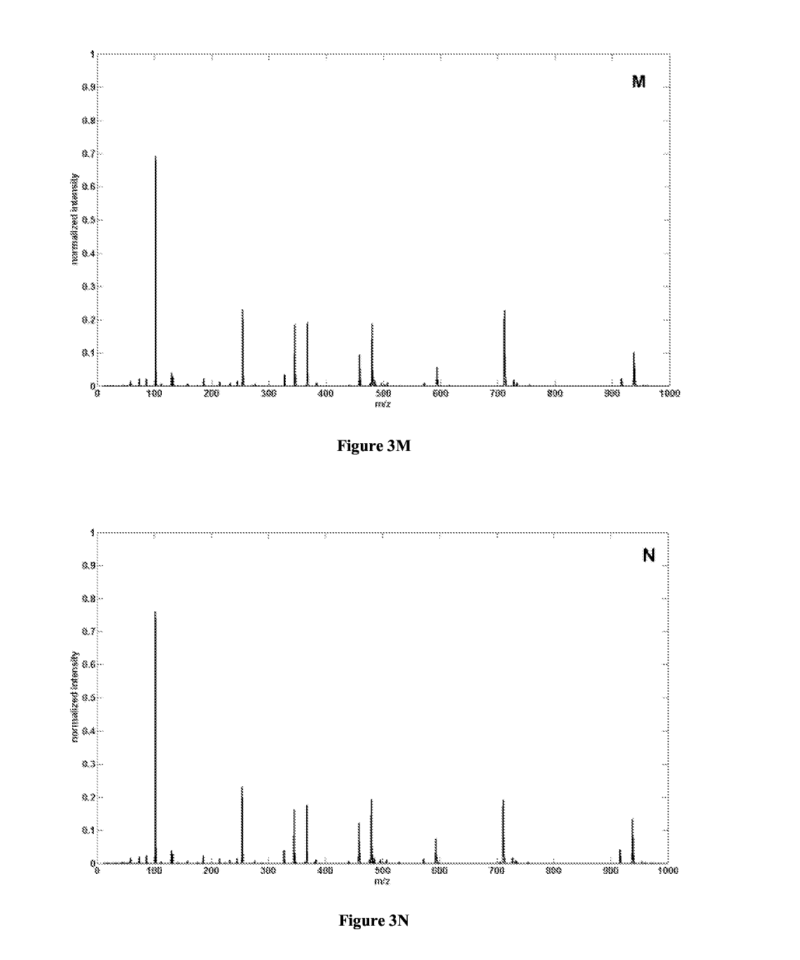

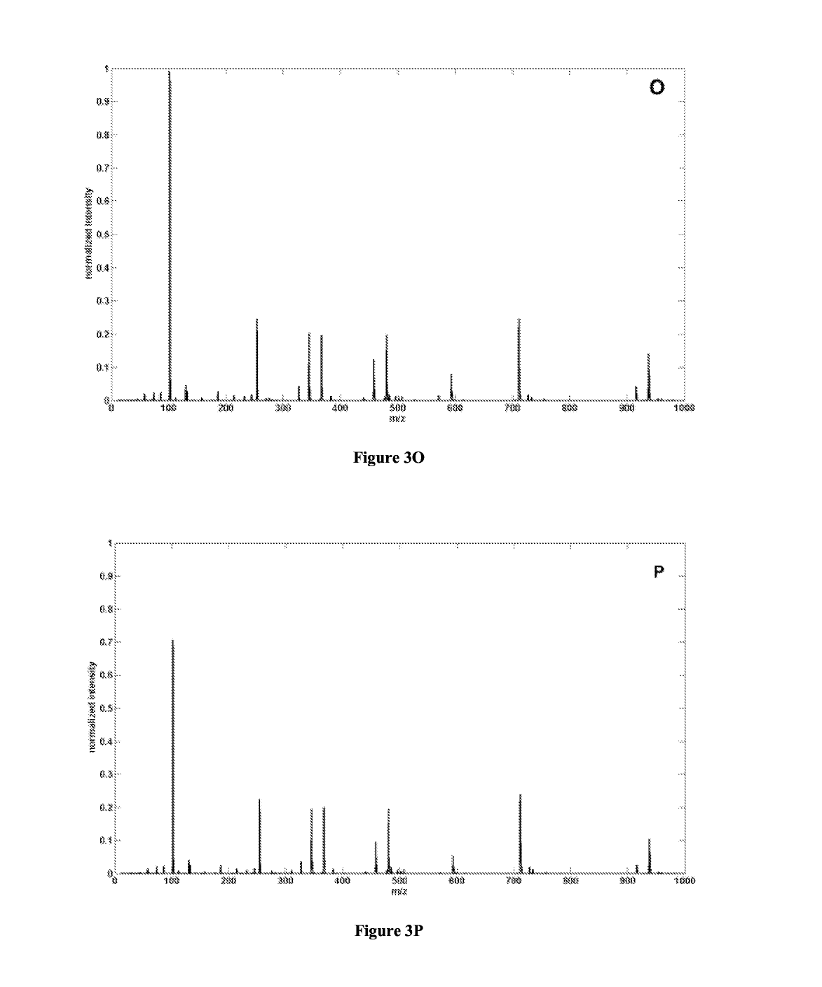

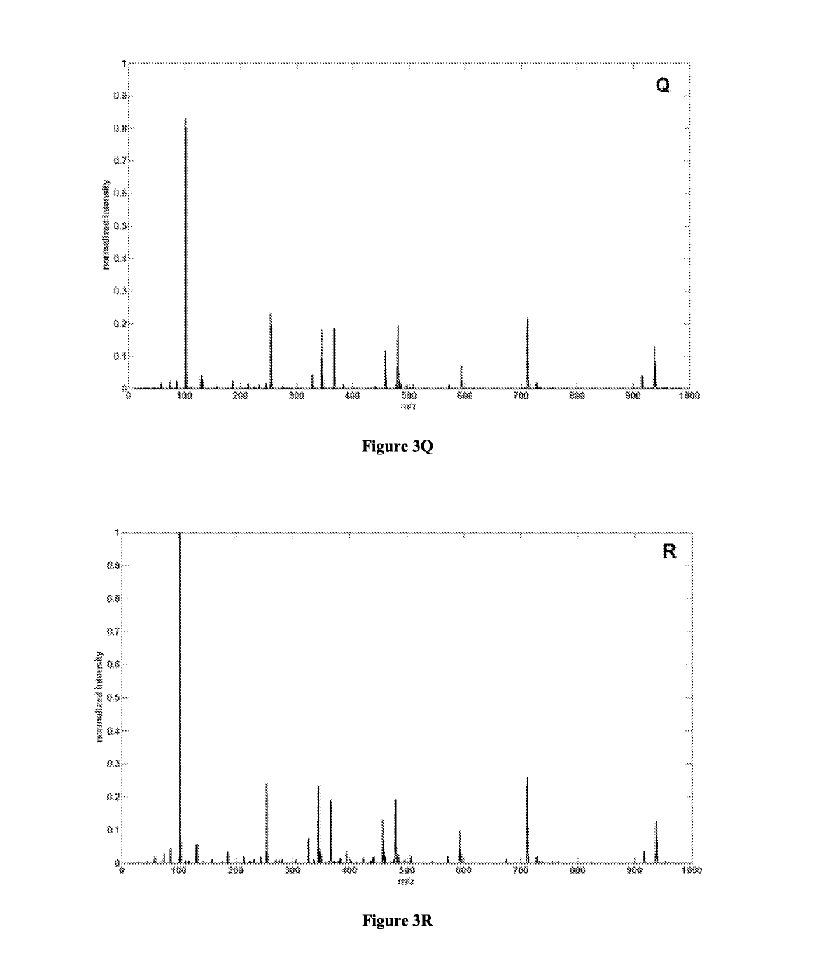

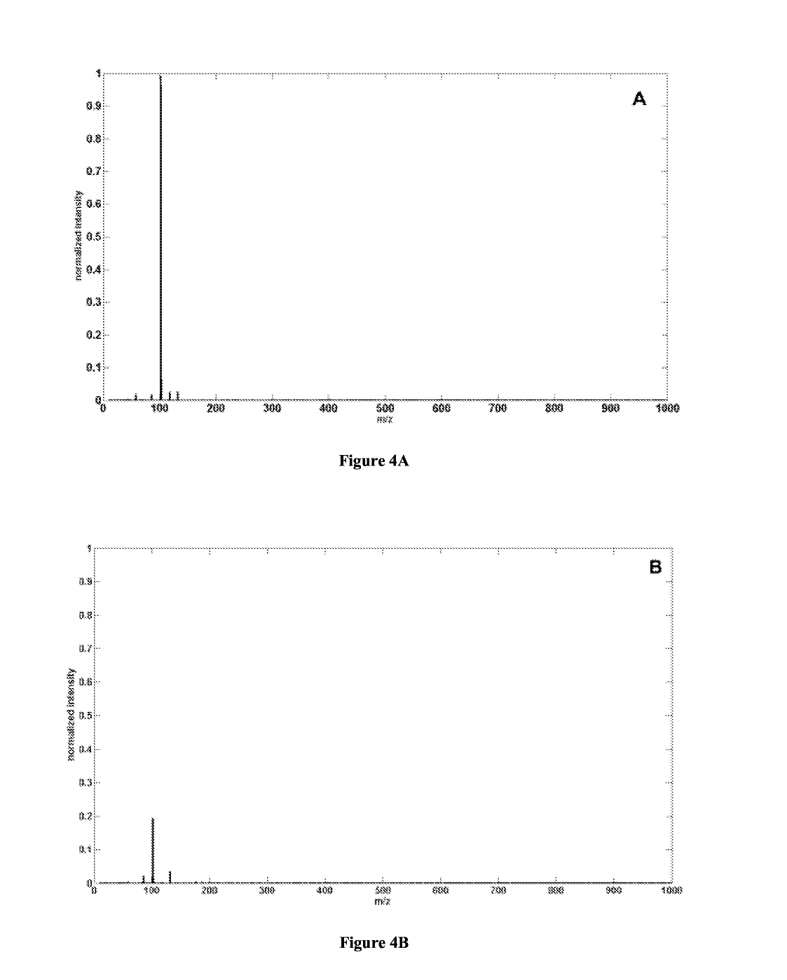

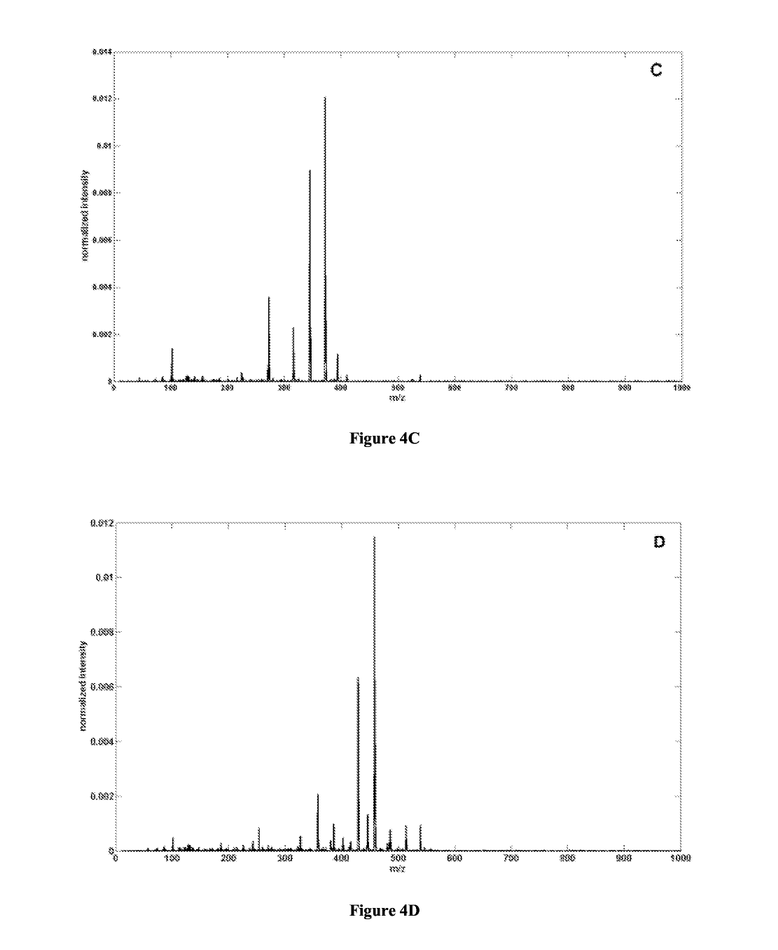

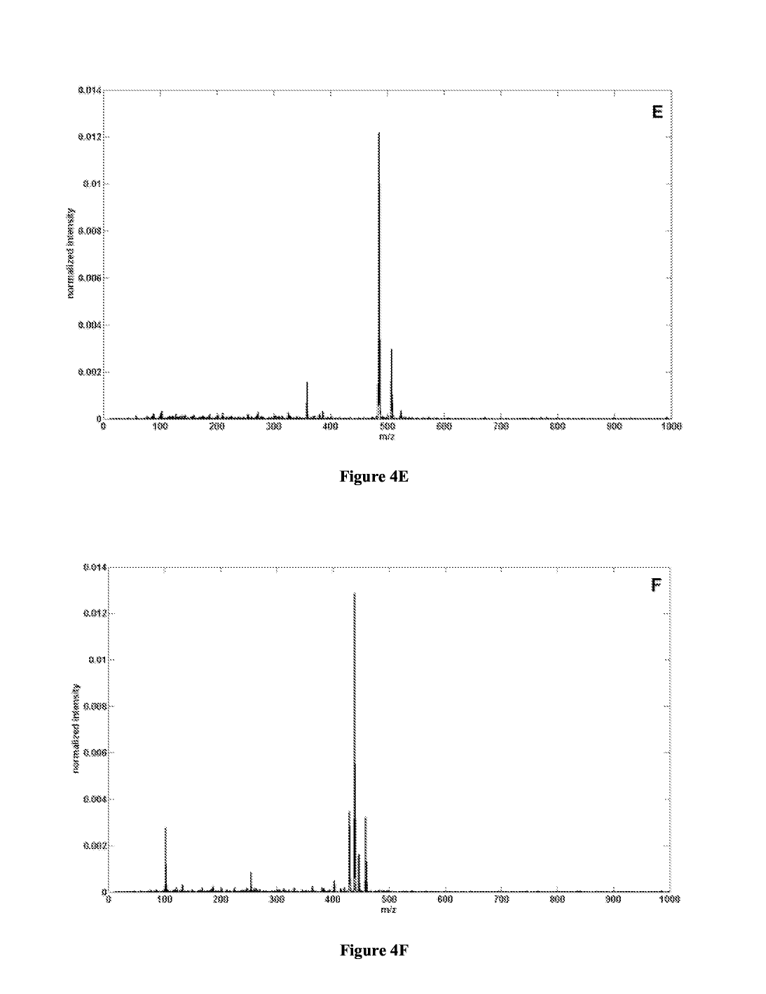

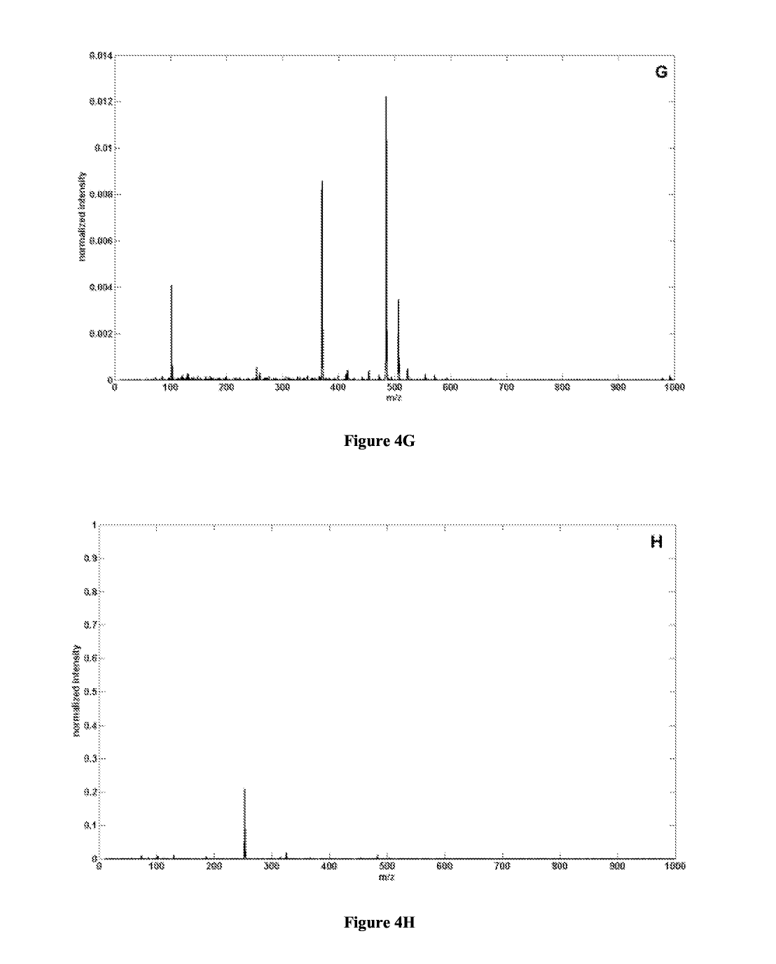

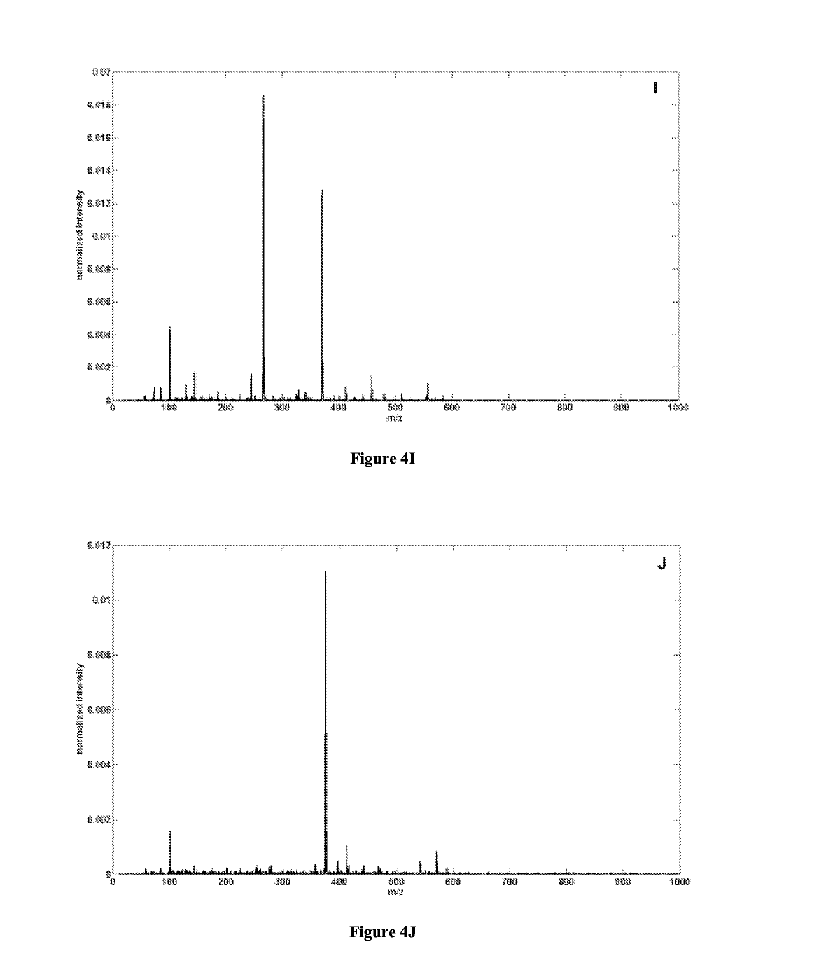

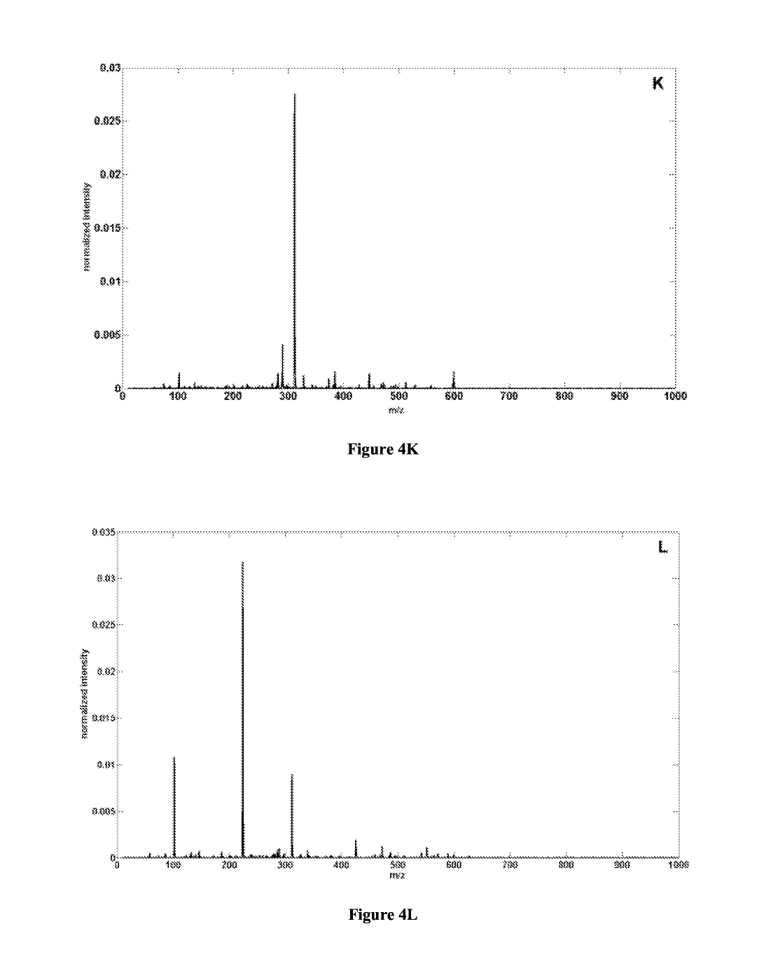

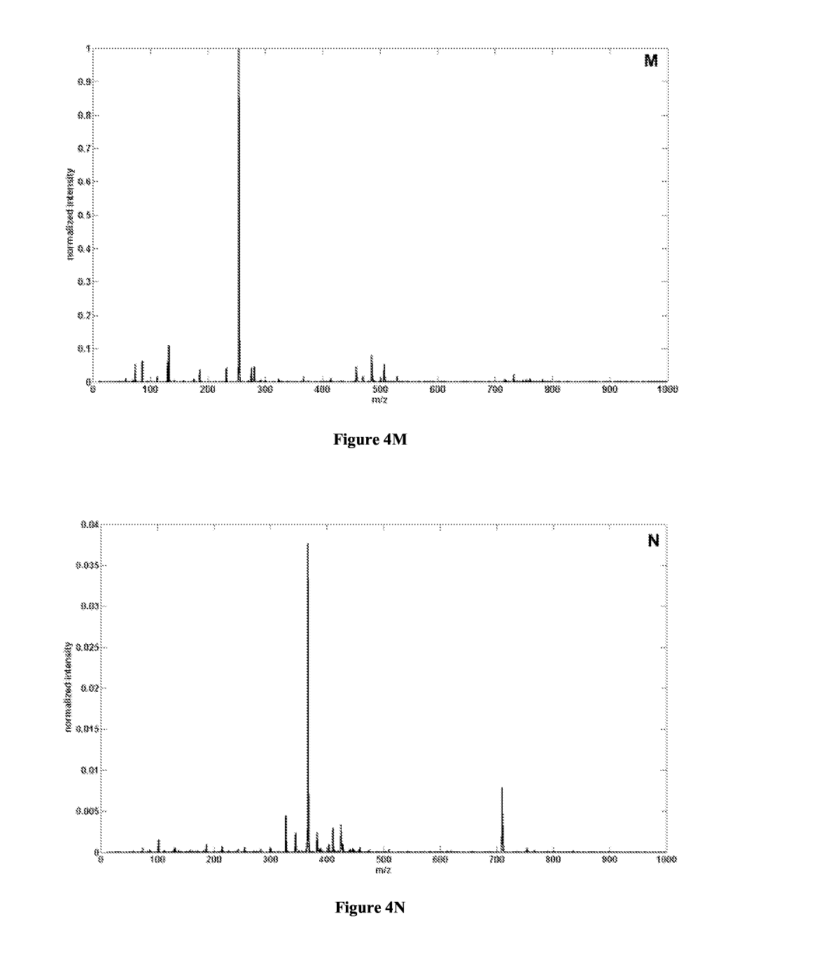

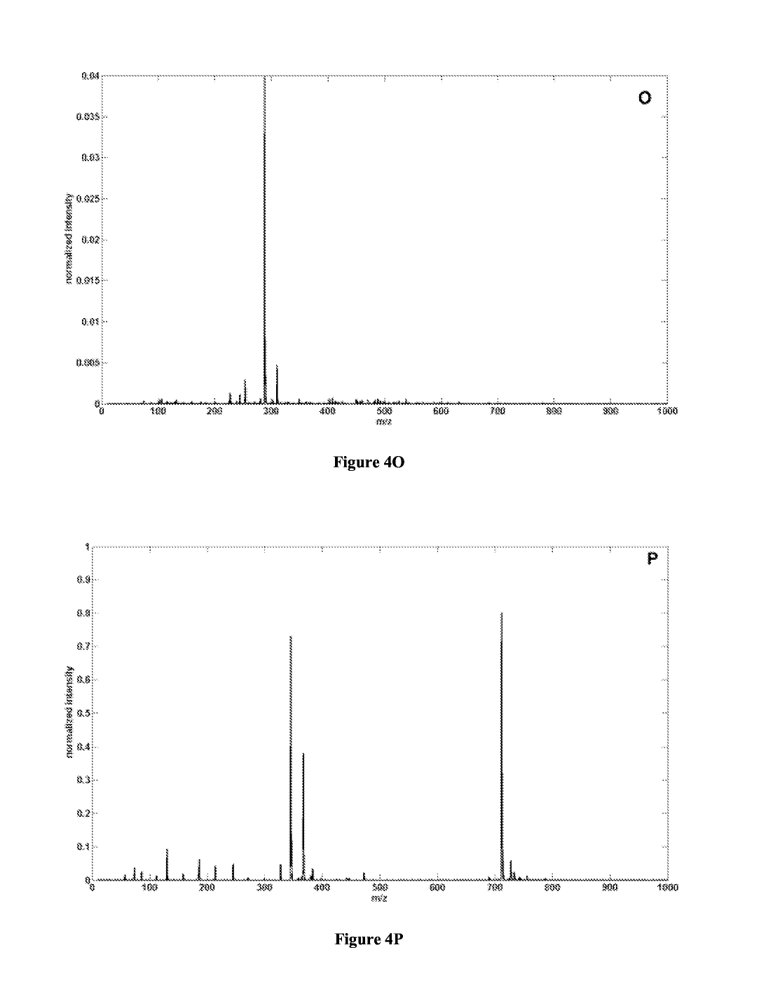

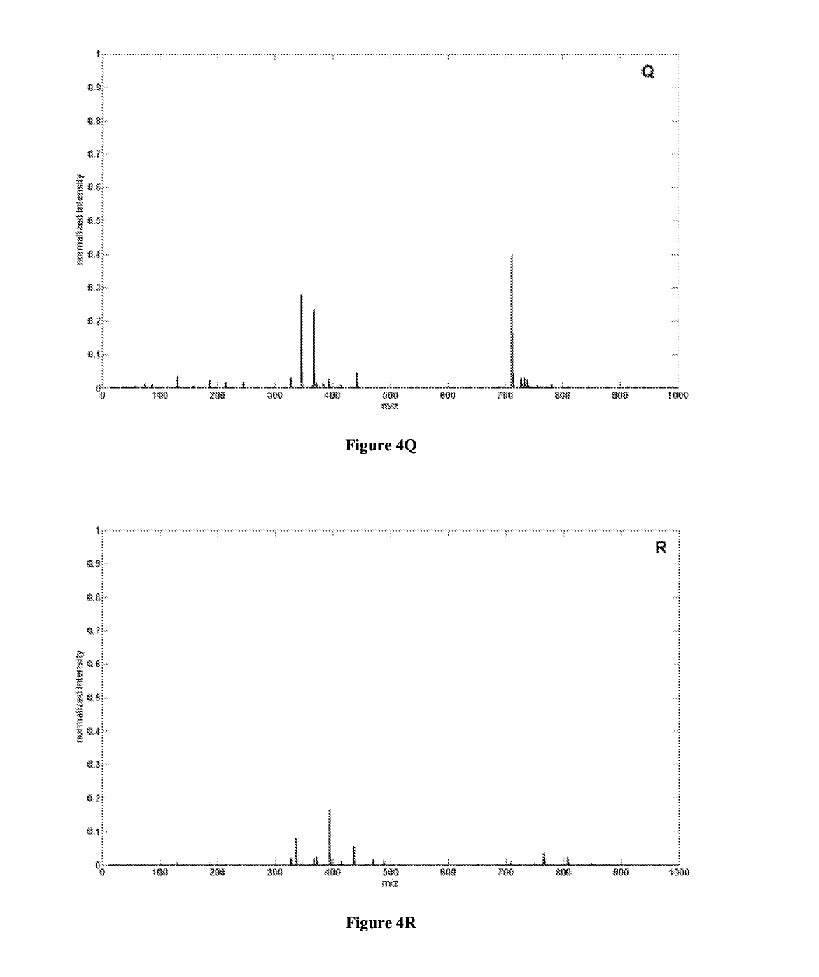

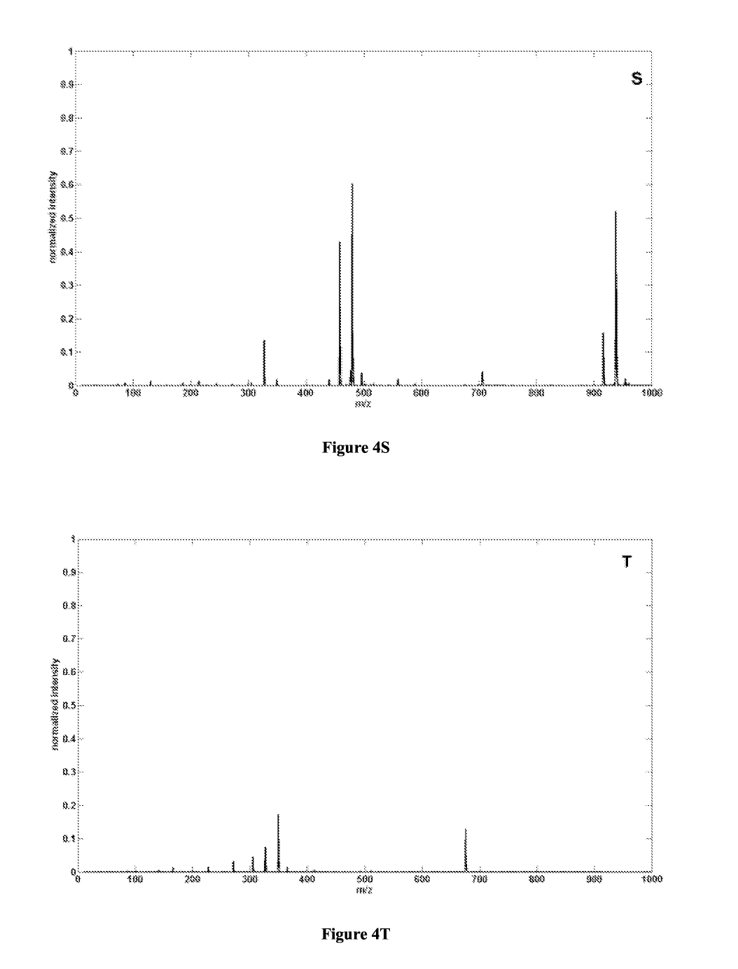

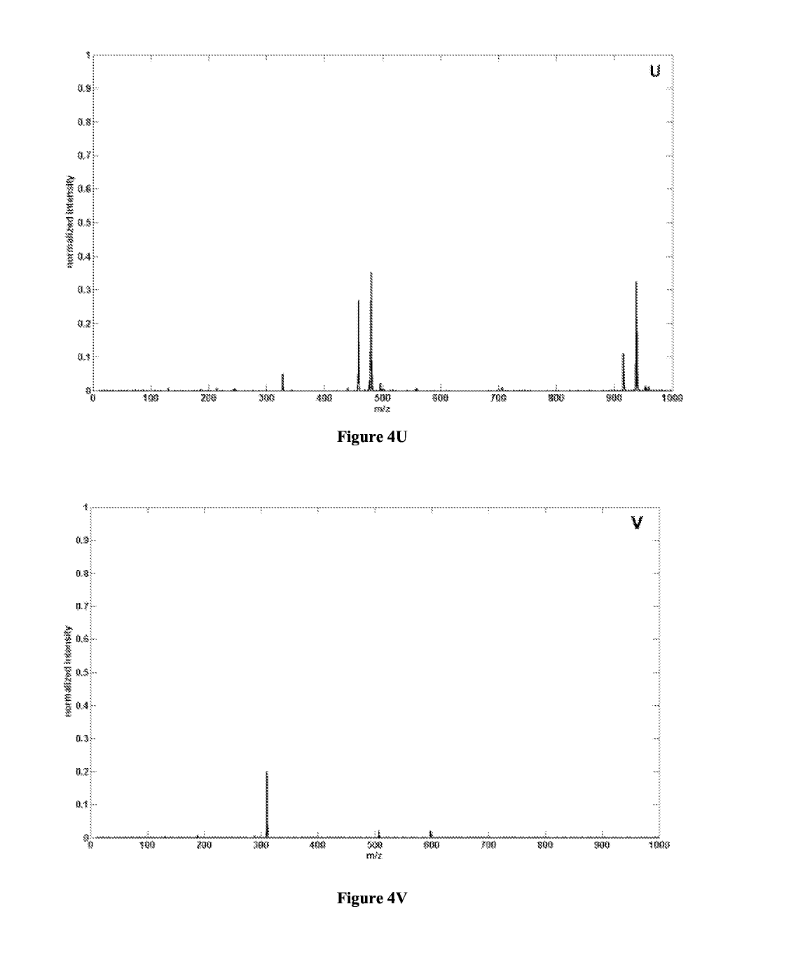

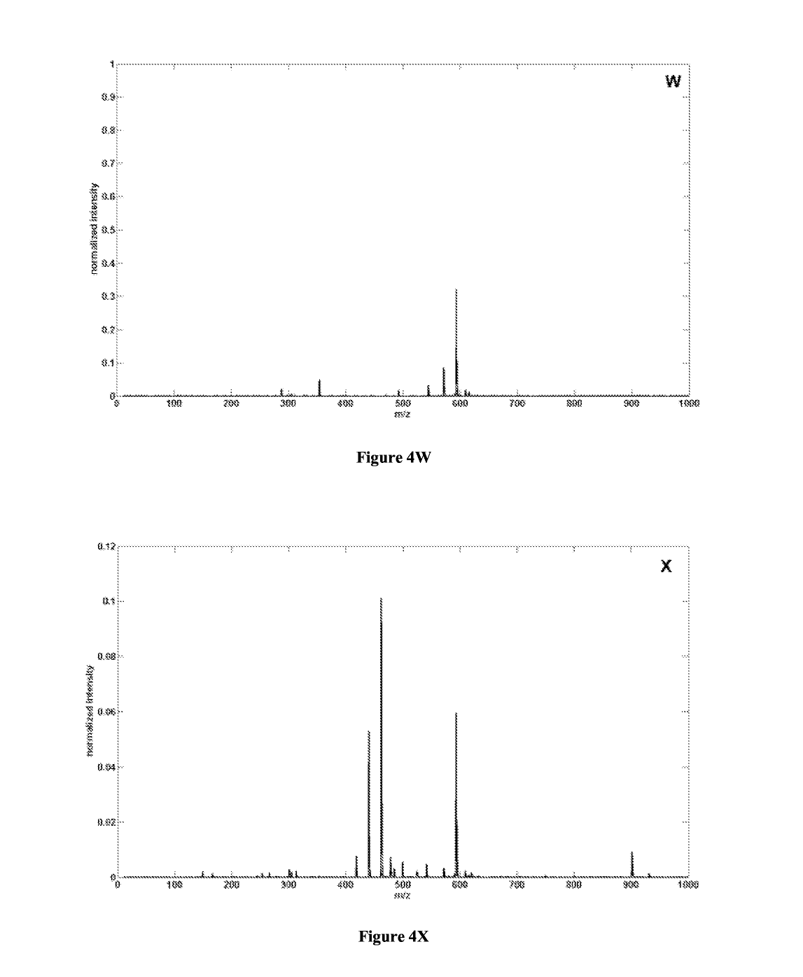

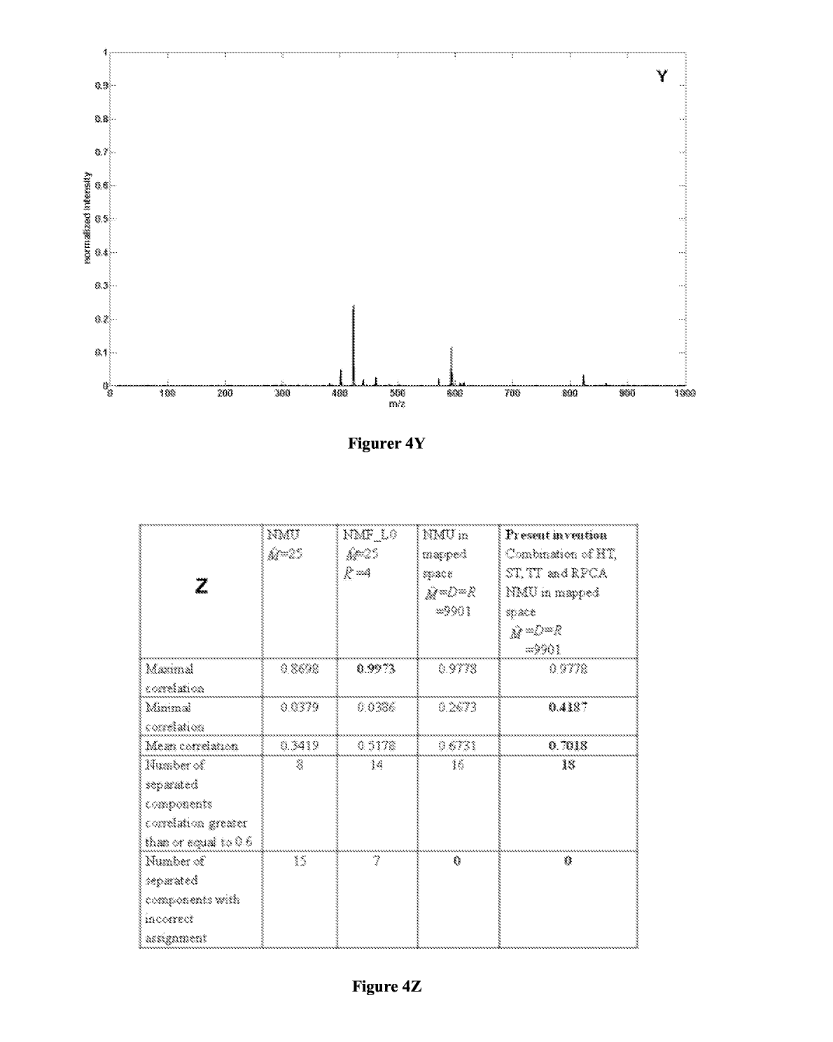

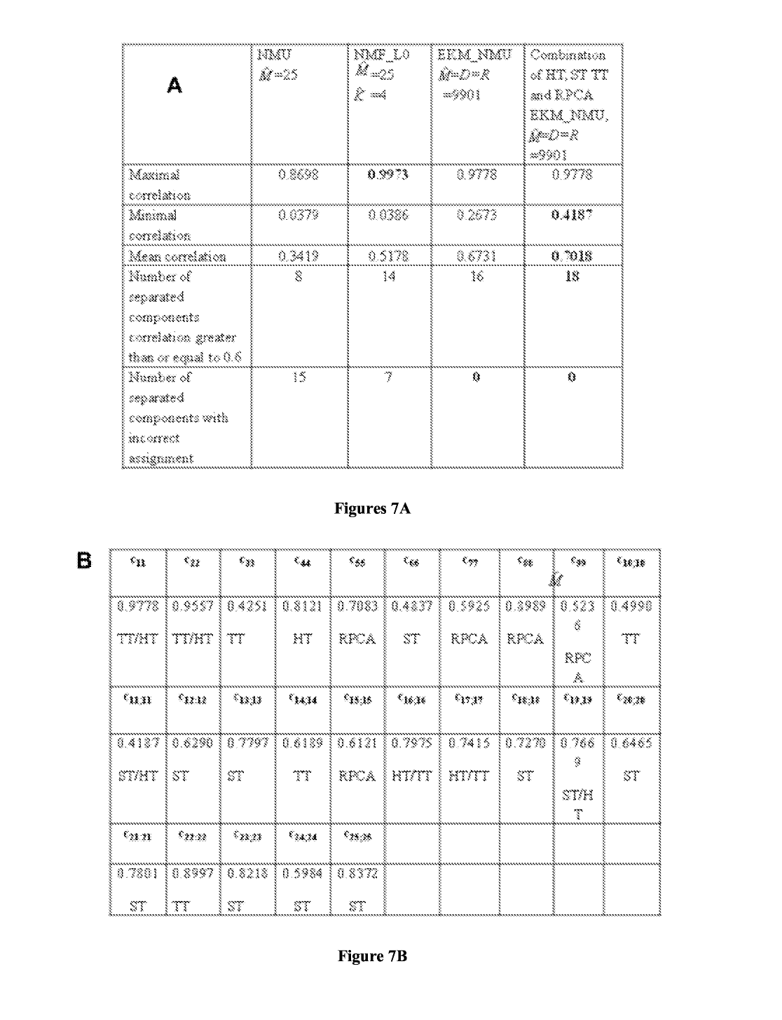

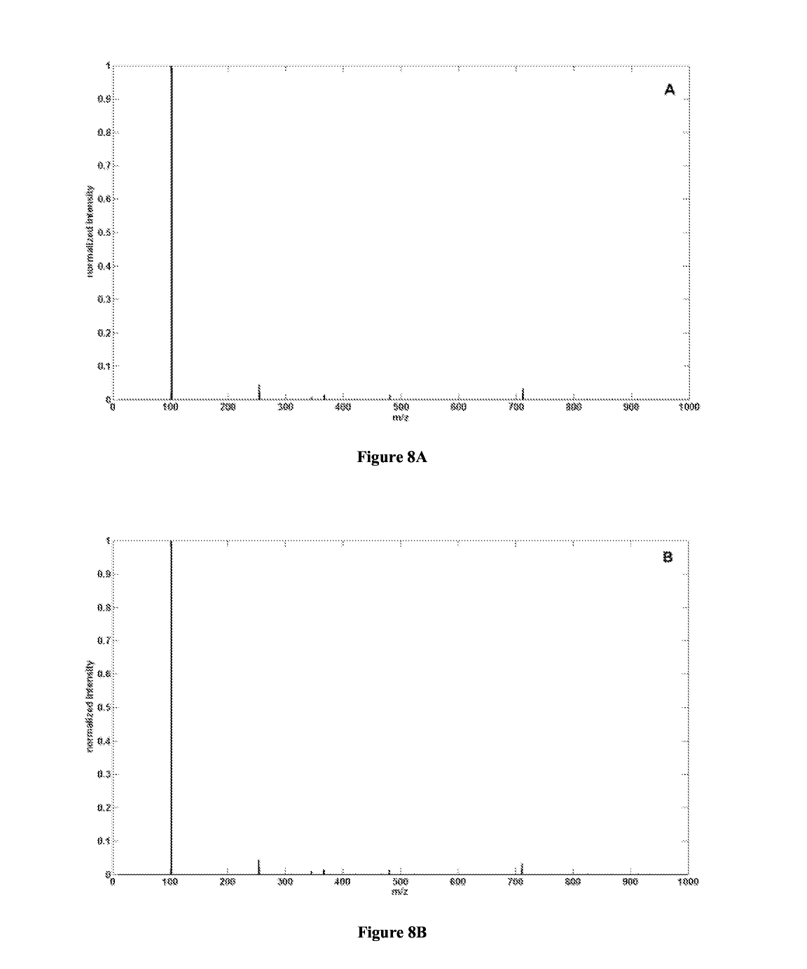

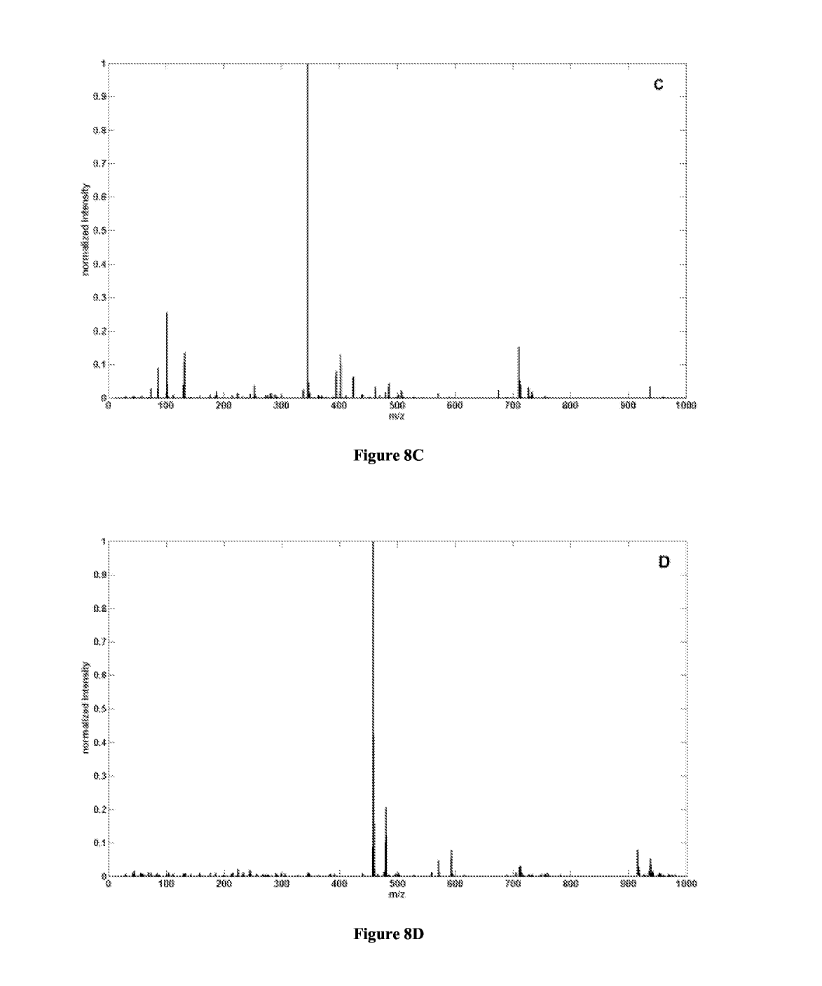

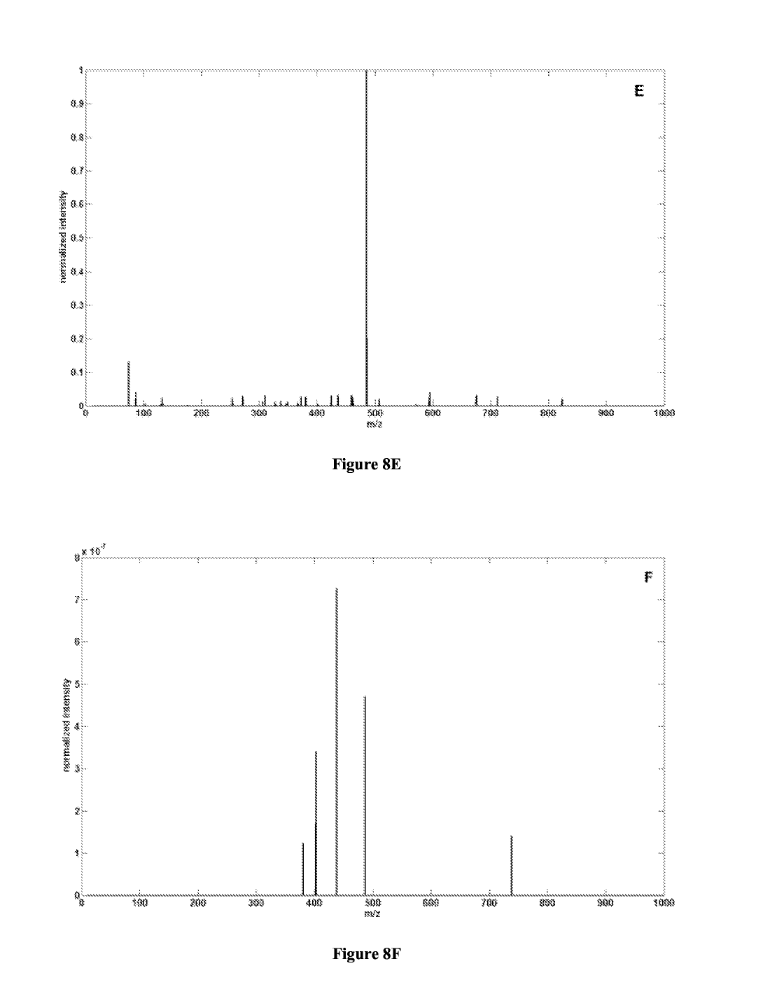

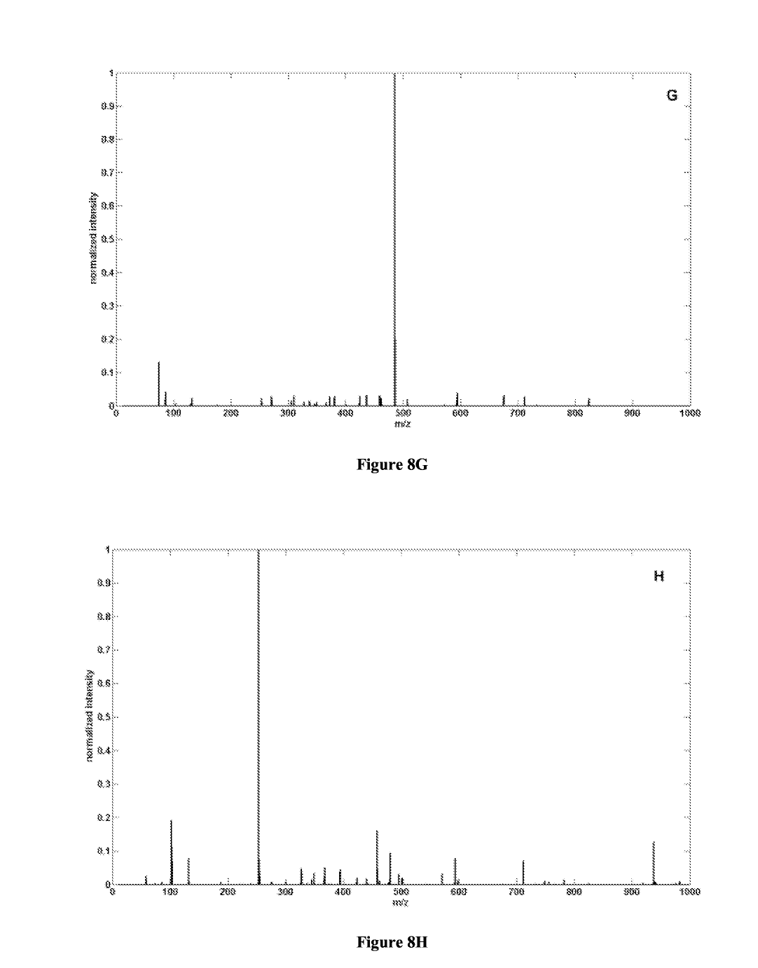

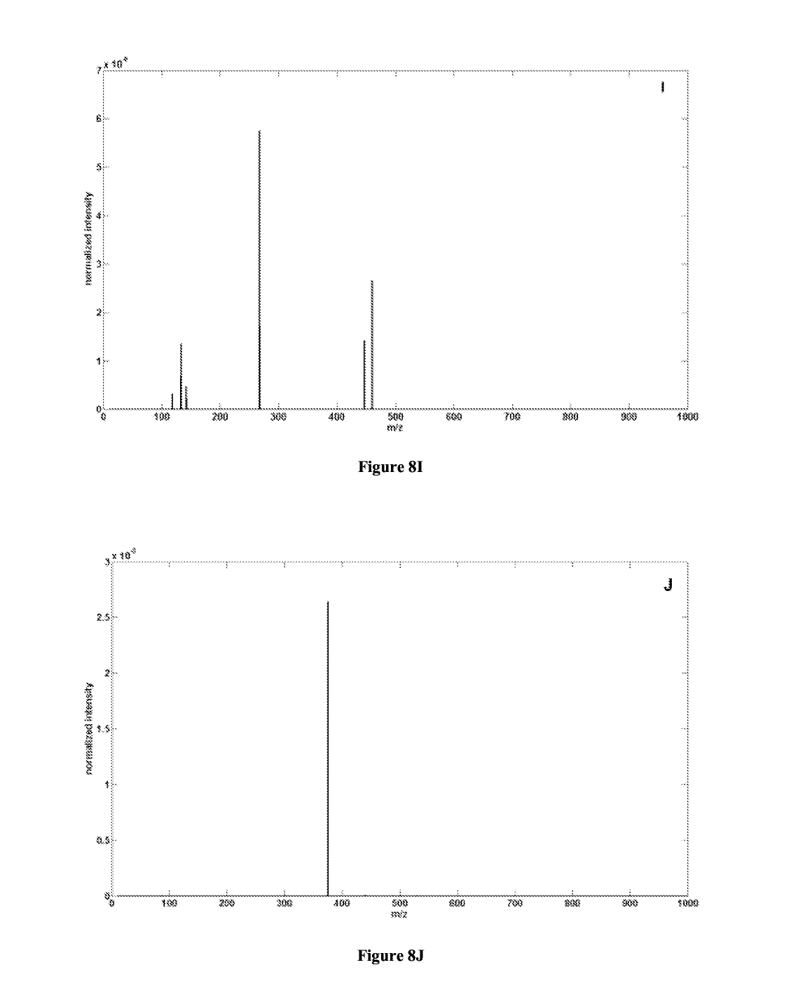

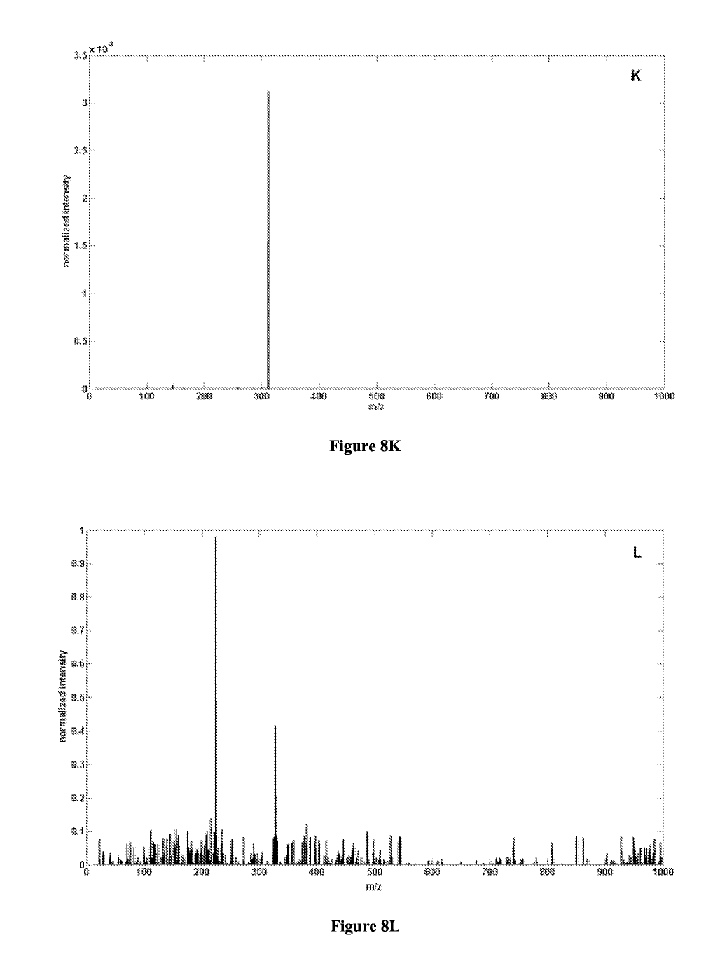

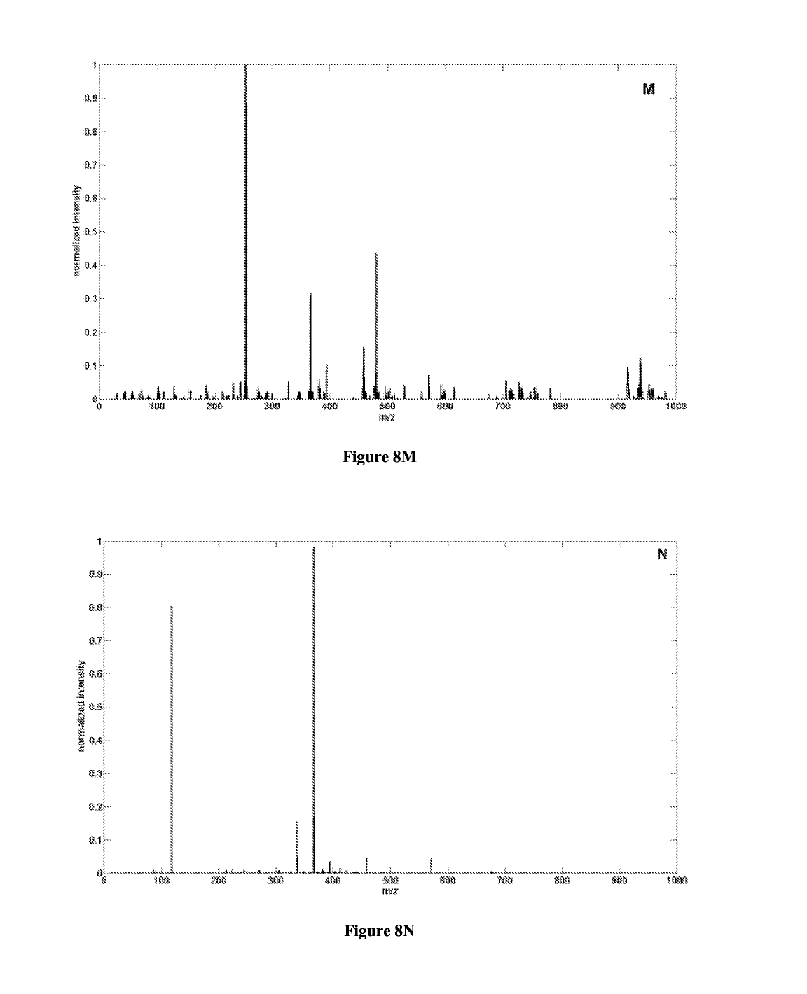

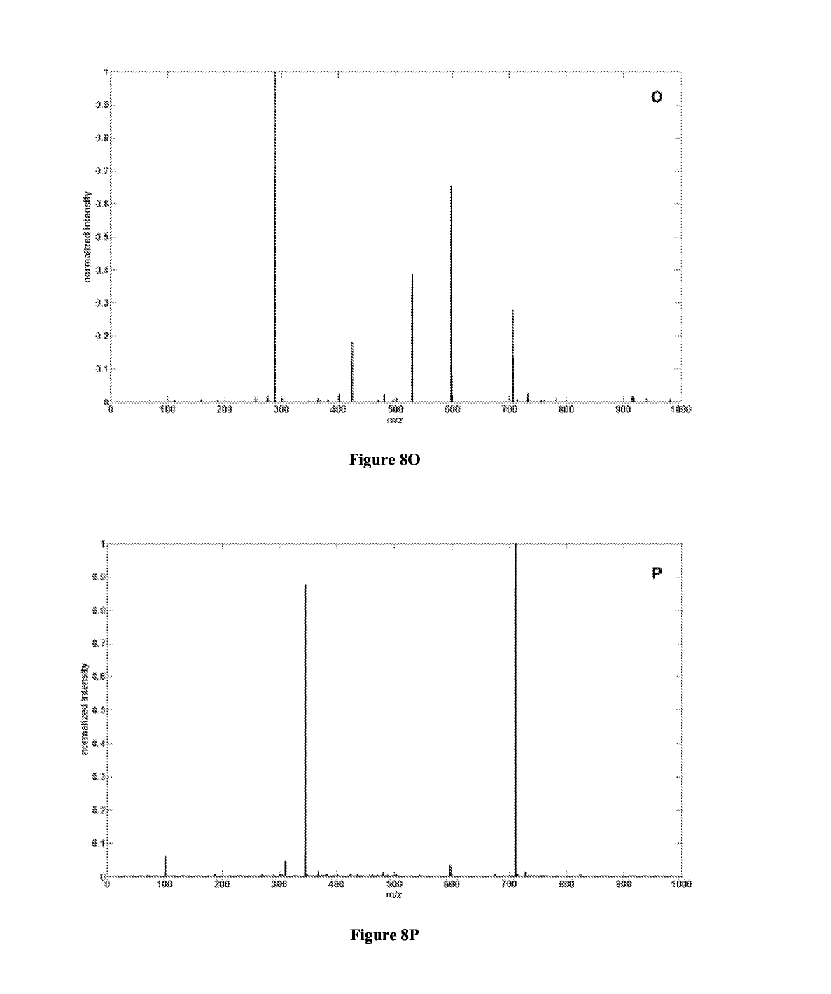

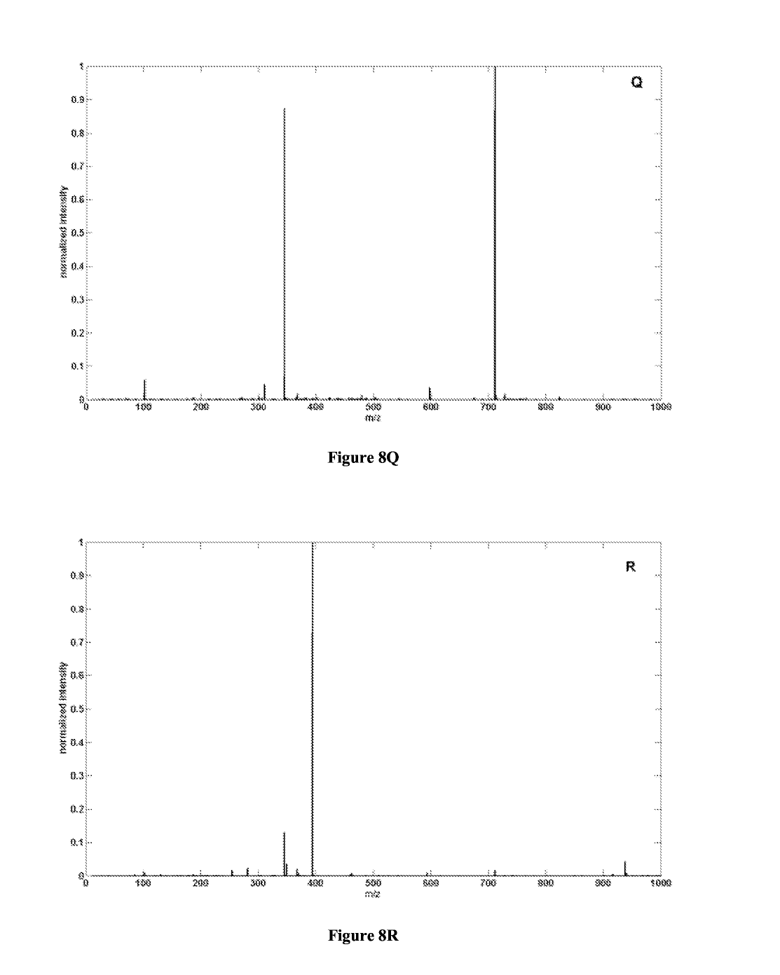

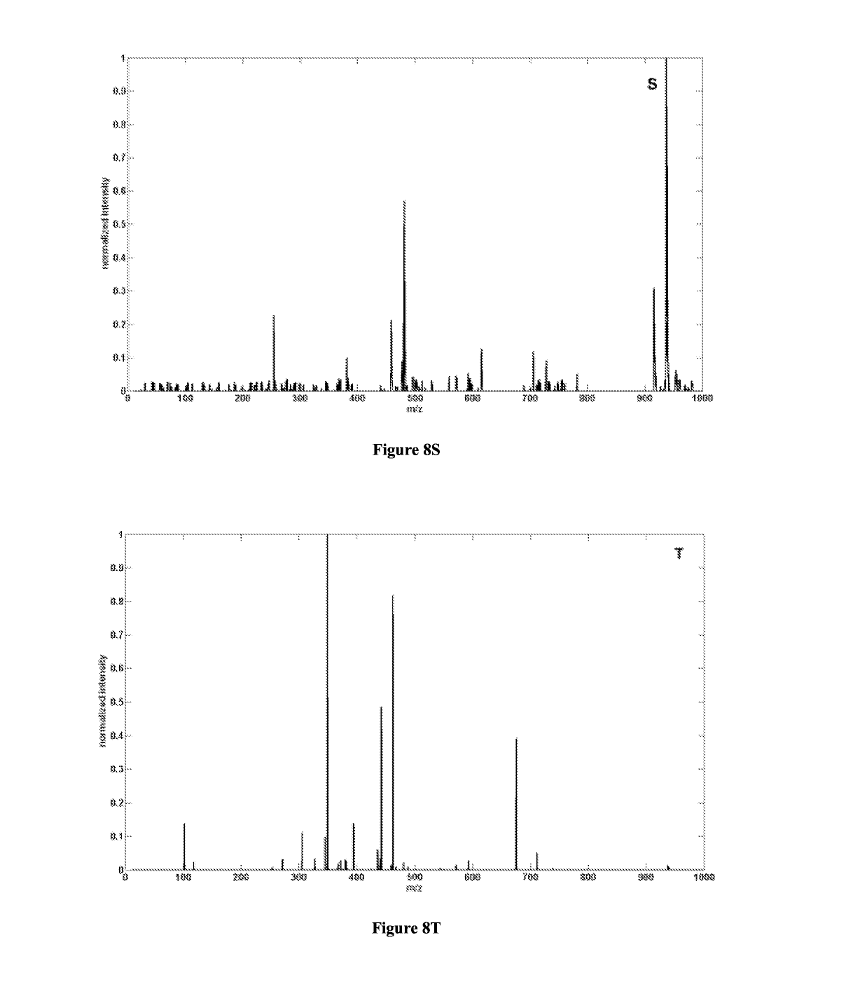

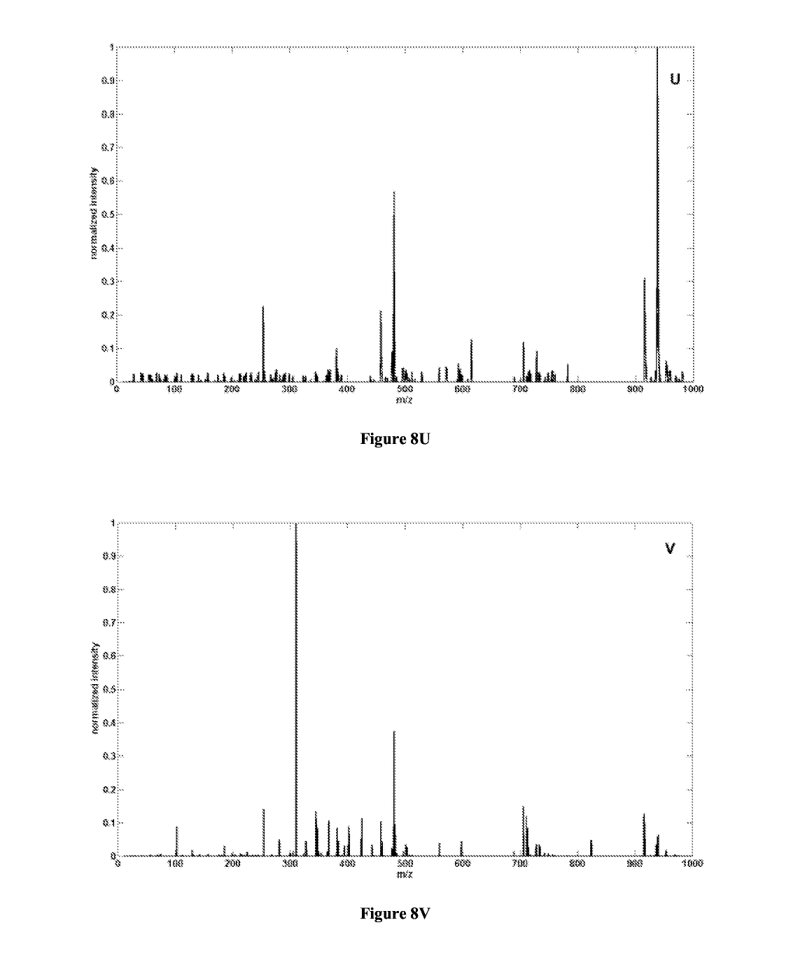

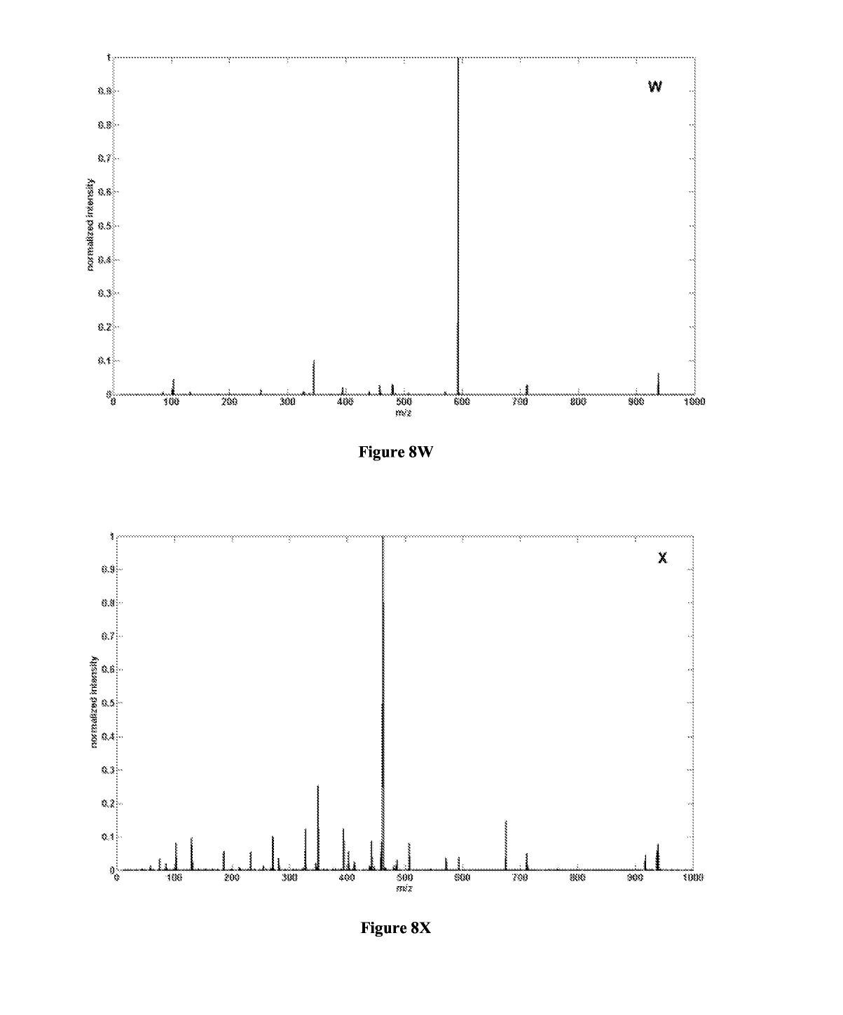

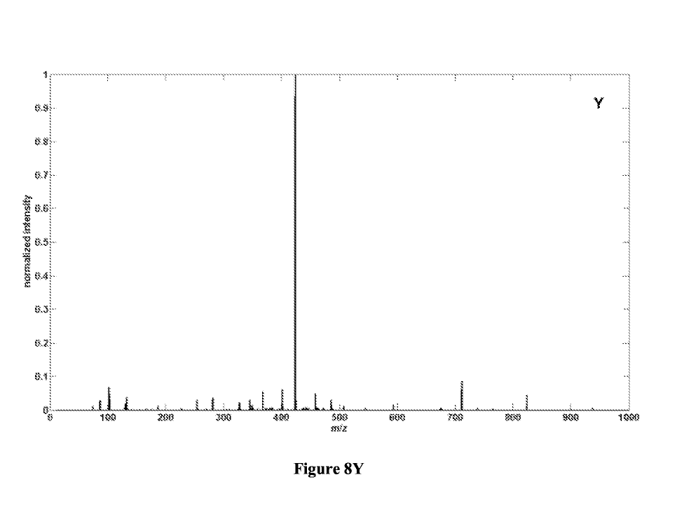

FIGS. 2 to 8 demonstrate experimental blind separation of 25 pure components mass spectra from 9 nonlinear mixtures mass spectra according to an embodiment of the present invention. FIG. 2 schematically describes nonlinear chemical reaction related to amine bond formation (synthesis of peptides). Chemical reaction has been performed according to the following procedure: L-Leucine (200 mg, 1.52 mmol) was dissolved in 5 mL of dry dimethylformamide (DMF) and solution was cooled to 0° C. N-methylmorpholine (NMM, 3.05 mmol, 337 μL) and isobutylchloroformate (IBCF, 3.34 mmol, 458 μL) were added. Aliquots of the reaction mixture (100 μL) were withdrawn every 30 minutes (t₀-t₈), solvent was evaporated and the residue dissolved in 1 mL of 0.1% formic acid (FA) in 50% MeOH. Aliquots (100 μL) were diluted with 400 μL of 0.1% FA in 50% MeOH and 10 μL were injected through autosampler on a column (Zorbax XDB C18, 3.5 lm, 4.6975 mm) at the flow rate of 0.5 mL/min. Mobile phase was 0.1% FA in water (solvent A) and 0.1% FA in MeOH (solvent B). Gradient was applied as follows: 0 min 40% B; 0-15 min 90% B; 12-15 min 90% B; 17.1 min 40% B; 17.1-20 min 40% B. FIGS. 3A to 3I show 9 chromatograms recorded during the reaction. FIGS. 3J to 3R show mass spectra of 9 nonlinear mixtures obtained by full integration of chromatograms shown correspondingly on FIGS. 3A to 3I. Thereby, electrospray ionization-mass spectrometry (ESI-MS) measurements operating in a positive ion mode were performed on a HPLC-MS triple quadrupole instrument equipped with an autosampler (Agilent Technologies, Palo Alto, Calif., USA). The desolvation gas temperature was 300° C. with flow rate of 8.0 L/min. The fragmentor voltage was 135 V and capillary voltage was 4.0 kV. Mass spectra were recorded in m/z segment of 10-2000. All data acquisition and processing was performed using Agilent MassHunter software. Pure components mass spectra generated during nonlinear reaction are shown in FIGS. 4A to 4Y. The library of pure components was generated by integration of each peak in the chromatogram and subsequent extraction of mass spectrum. FIG. 4Z shows correlation matrix of the pure components mass spectra, whereas pairs of pure components are identified with normalized correlation coefficient above 0.1. There are 30 such pairs. Moreover, pure components 1 and 2, pure components 16 and 17 and pure components 19 and 21 have normalized correlation coefficient above 0.97 and, consequently, they are impossible to be distinguished. In addition to that, pure components 5 and 7 have normalized correlation coefficient above 0.78. Thus, they are also expected to be very hard to discriminate. Normalized correlation coefficients for other pairs vary between 0.1 and 0.44 and that makes the nonlinear underdetermined blind source separation problem comprised of correlated pure components to be very hard. What makes solution of such hard problem possible is sparseness of the pure components mass spectra in support and amplitude. To this end, FIG. 5A shows estimated probability that value of the pure component mass spectra is zero for 25 pure components shown in FIGS. 4A to 4Y. As can be seen 22 out of 25 pure components have zero amplitudes at 40% to 75% of their support. FIG. 5B show most expected value (mean) of exponential distribution estimated by fitting exponential distribution to amplitude histograms. They were estimated for 25 pure components in the range (0, 1] within intervals of the 0.01 width. As seen from FIG. 5B estimated mean values are in the range [0.0012, 0.0015]. FIG. 5C shows probability that amplitude of the pure components mass spectra occurs in interval [0, A], such that 0.01≦A≦1. That is average estimate across 25 pure components. FIGS. 6A to 6Y show estimated histograms (stars) and exponential probability density functions (squares) calculated with the mean values from FIG. 5B. It can be seen that approximation is very good. It can be seen from FIG. 5C that 47% of the amplitudes are in the interval (0, 0.01]. That is in agreement with results shown in FIG. 5B. It can be estimated from FIG. 5A that average probability that value of the pure component is zero equals 0.4996. Thus, in overall 97% of the amplitudes are in the interval [0, 0.01]. Nevertheless, theoretical calculations based on exponential probabilistic prior show that for most expected value 0.01 there is still 29% chance that amplitude of the pure components mass spectra will be greater than 0.01. That justifies inclusion of the second order terms in truncated Taylor expansions in [IV]. FIG. 7A shows best results obtained by present invention: sparseness and nonnegativity constrained factorization of the mixture data matrices in mapped space according to [XIII], [XIV], [XV] and [XVI] and choosing the best result according to [XVII] to [XXV]. Thereby, assumed number of components in mapped space was \hat{M}=9901 (that is overall number of m/z lines in mass spectra). For comparison purpose results of factorizations of mixture data matrix [IV] by means of nonnegative matrix underapproximation (NMU) algorithm, nonnegative matrix factorization algorithm constrained with l₀ quasi-norm (NMF_L0) are also reported in FIG. 7A. Assumed number of components for NMU and NMF_L0 algorithms was \hat{M}=25, while number of overlapped components for the NMF_L0 algorithm was \hat{K}=4. FIG. 7A also presents result for NMU algorithm applied to mapped matrix of mixtures mass spectra (no preprocessing related to error reduction has been done) with assumed number of the components in mapped space \hat{M}=9901. Results were compared in term of five criteria: maximal, minimal and mean correlation between separated and pure components from the library, number of separated components with correlation greater than or equal to 0.6 and number of incorrectly assigned components. The following conclusions are drawn from FIG. 7A: (i) proposed invention significantly increases number of separated components that are correlated with pure components from the library with correlation above 0.6; (ii) number of incorrectly assigned component is reduced to zero; (iii) mean correlation of all separated components is 0.7; (iv) minimal correlation is increased to 0.42 and that is especially important for separating pure components (metabolites) that are present in the mixture in small concentration and can be covered by dominant components. FIG. 7B shows results obtained by present invention in term of correlation coefficient between pure components from the library and assigned separated components. Thereby, error-reduction technique used in preprocessing step in obtaining separated component is also presented, whereas HT stands for hard thresholding, ST stands for thresholding, TT stands for trimmed threshodling and RPCA stands for robust principal component analysis. Finally, FIGS. 8A to 8Y show mass spectra of the components separated by proposed invention and assigned to the pure components from the library using highest correlation criterion. Visual comparison with corresponding images shown in FIGS. 4A to 4Y illustrates quality of separation. The most important conclusion, however, is that separated components are assigned to the proper components from the library.

The present invention relates to the field of mass spectrometry. More specifically, the invention relates to blind separation of nonnegative correlated pure components from smaller number of nonlinear mixtures spectra recorded by mass spectrometer. Proposed invention separates pure components by means of sparseness and nonnegativity constrained factorization of empirical kernel map-based mappings of matrices obtained by preprocessing matrix of mixtures mass spectra. Thus, data matrix comprised of recorded mixtures mass spectra [II]/[IV] is decomposed by means of robust principal component analysis into low-rank and sparse matrices prior mapping and factorization. It is also preprocessed by hard, soft and trimmed thresholding. The enabling constraint for solution of nonlinear underdetermined blind source separation problem is sparseness of the pure components mass spectra in support and amplitude. That is demonstrated in FIGS. 4A to 4Y, where mass spectra of pure component generated by nonlinear chemical reaction of peptide bond formation are shown, and in FIGS. 5A to 5C and FIGS. 6A to 6Y which show good fit of the empirical pure components mass spectra to mixed state probabilistic model [III] with exponential prior on amplitudes of the pure components. By large probability, this joint sparseness eliminates monomials of order higher than 2 in truncated Taylor expansions [IV]. Thus, in case of mapped preprocessed matrices [IX], [X], [XI] and [XII] benefits of new mixtures generated by nonlinear mapping will prevail over side effects caused by higher-order terms that, themselves, represent new components that are correlated with the pure components. If number of pure components M and maximal number of overlapped pure components K are known it is possible to estimate number of components and maximal number of overlapped components in mapped space according to [XIII], [XIV], [XV] and [XVI]. They are respectively given as: \hat{M}≈2M+M(M−1)/2 and \hat{K}≈2K+K(K−1)/2. The alternative strategy is to exhaust the whole space by extracting D components, whereas D≦R. In any case, components separated in [XIII], [XIV], [XV] and [XVI] are correlated with the reference components in the library in [XVII], [XVIII], [XIX] and [XX]. Separated components are assigned to the pure components from the library using highest correlation criterion. In this regard, FIGS. 8A to 8Y show mass spectra of components separated from mixtures spectra of nonlinear chemical reaction of peptide formation by means of proposed invention, whereas values of normalized correlation coefficients are presented in FIG. 7B.

Constraint on sparseness of pure components in terms of support and amplitude is what enables solution of the related nonlinear underdetermined blind source separation problem comprised of nonnegative dependent (correlated) sources (pure components). That is distinction with respect to other algorithms that comprise state-of-the-art in nonlinear blind source separation and that either use other constraints or use sparseness constraint related to support only.

The present invention can be applied to the identification of compounds in the pharmaceutical industry, in the chemical synthesis of new compounds. It can also be applied in the food quality inspection and environment protection through pollution inspection. Another application of proposed invention is in commercial software packages, as built in computer code, that are used for analysis and identification of chemical compounds. Possibly the most important application of the present invention is in instrumental diagnostics, determination and identification of biomarkers present in biological fluids (urine, blood plasma, cerebrospinal fluid, saliva, amniotic fluid, bile, tears, etc) or tissue extracts; detection of pathologies (genetically determined diseases), detection of patients with predisposition for certain disease, monitoring the response of the organism to the action of pharmaceuticals, pathogens or toxic compounds (wars, natural or ecology disasters).

What is claimed is:

1. A method for blind separation of nonnegative correlated pure components from smaller number of nonlinear mixtures mass spectra by using robust principal component analysis, trimmed thresholding, hard thresholding and soft thresholding for preprocessing of experimental data matrix of mixtures mass spectra; empirical kernel map-based nonlinear mapping of preprocessed matrices onto reproducible kernel Hilbert space, sparseness and nonnegativity constrained factorization of mapped matrices, correlation of separated components with the reference components from the library and assignment of the separated components to the pure components from the library using maximal correlation criterion, comprising the following steps:
recording and storing the mixtures data X, where X is nonnegative data matrix comprised of N≧2 rows that correspond to mixture mass spectra and R columns that correspond to observations at different mass-to-charge (m/z) ratios,
scaling the mixture data matrix by maximal element of X, xₘₐₓ:
X=X/xₘₐₓ [I]
that yields new data matrix X such that 0≦xₙᵣ≦1, n=1, . . . , N, and r=1, . . . , R,
representing scaled mixture data matrix in [I] by nonlinear mixture model:
X=f(S) [II]
where S stands for an unknown nonnegative matrix comprised of M>N rows {sₘ}ₘ₌₁^{M} that correspond with pure components mass spectra and R columns that correspond with observations at different m/z ratios; f(S) implies that nonlinear mapping is performed column-wise: xᵣ=f(sᵣ) r=1, . . . , R, whereas f(sᵣ)=[ƒ(sᵣ) . . . ƒ_{N}(sᵣ)]^{T} and {ƒₙ: R₀₊^{M}→R₀₊}ₙ₋₁^{B}. Scaling [I] implies that 0≦sₘᵣ≦1, m=1, . . . , M and r=1, . . . , R,
using mixed state probabilistic model for the amplitudes of the pure components mass spectra sₘᵣ:
p(sₘᵣ)=ρₘδ(sₘᵣ)+(1−ρₘ)δ*(sₘᵣ)ƒ(sₘᵣ) [III]
where δ(sₘᵣ) is an indicator function and δ*(sₘₜ)=1−δ(sₘᵣ) is its complementary function, ρₘ stands for probability that sₘᵣ=0. Thus, 1−ρₘ stands for probability that sₘᵣ>0. ƒ(sₘᵣ) is continuous probability density function that models sparse probability distribution of the amplitude sₘᵣ.
representing [II] by using truncated Taylor expansion:
$$X = {G}_{s} S + {G}_{{s}^{2}} [ {\{ {s}_{{m}_{1}} {s}_{{m}_{2}} \}}_{{m}_{1} , {m}_{2} = 1}^{M} ] + HOT \qquad [ IV ]$$
where {sₘ_{₁}sₘ_{₂}}ₘ_{₁}_{,m}_{₂}₌₁^{M} stand for second order monomials that are cross-products between pure components {sₘ}ₘ₌₁^{M}, Gₛ and Gₛ_{²}, are matrices of appropriate dimensions and HOT stands for higher-order terms that include monomials of order greater than 2,
apply robust principal component analysis to X in [IV] to obtain:
X=A+E [V]
where
$$A \approx {G}_{s} S + {G}_{{s}^{2}} [ {\{ {s}_{{m}_{1}} {s}_{{m}_{2}} \}}_{{m}_{1} , {m}_{2} = 1}^{M} ]$$
stands for low-rank matrix composed of linear combination of original pure components and linear combination of second order monomials that represent new components correlated with the original ones, and E≈HOT stands for sparse matrix that represents error terms associated with higher-order monomials,
apply hard threshodling operator to X in [IV] to obtain:
$$B \approx {G}_{s} S + {G}_{{s}^{2}} [ {\{ {s}_{{m}_{1}} {s}_{{m}_{2}} \}}_{{m}_{1} , {m}_{2} = 1}^{M} ] \qquad [ VI ]$$
where B stands for hard thresholded version of X in [IV],
applying soft thresholding operator to X in [IV] to obtain:
$$C \approx {G}_{s} S + {G}_{{s}^{2}} [ {\{ {s}_{{m}_{1}} {s}_{{m}_{2}} \}}_{{m}_{1} , {m}_{2} = 1}^{M} ] \qquad [ VII ]$$
where C stands for soft thresholded version of X in [IV],
applying trimmed thresholding operator to X in [IV] to obtain:
$$D \approx {G}_{s} S + {G}_{{s}^{2}} [ {\{ {s}_{{m}_{1}} {s}_{{m}_{2}} \}}_{{m}_{1} , {m}_{2} = 1}^{M} ] \qquad [ VIII ]$$
where D stands for trimmed thresholded version of X in [IV],
using empirical kernel map for nonlinear mapping of A in [V] onto reproducible kernel Hilbert space:
$$Ψ ( A ) = [ \begin{matrix}κ ( {a}_{1} , {v}_{1} ) & … & κ ( {a}_{R} , {v}_{1} ) \\ … & … & … \\ κ ( {a}_{1} , {v}_{D} ) & … & κ ( {a}_{R} , {v}_{D} )\end{matrix} ] \qquad [ IX ]$$
where κ(aᵣ,v_{d}), r=1, . . . , R and d=1, . . . , D stands for positive symmetric kernel function and v_{d}, d=1, . . . , D stand for basis vectors that approximately span the same space as the vectors: aᵣ, r=1=, . . . , R.
using empirical kernel map for nonlinear mapping of B in [VI] onto reproducible kernel Hilbert space:
$$Ψ ( B ) = [ \begin{matrix}κ ( {b}_{1} , {v}_{1} ) & … & κ ( {b}_{R} , {v}_{1} ) \\ … & … & … \\ κ ( {b}_{1} , {v}_{D} ) & … & κ ( {b}_{R} , {v}_{D} )\end{matrix} ] \qquad [ X ]$$
where interpretation of Ψ(B) is equivalent to those of Ψ(A) in [IX],
using empirical kernel map for nonlinear mapping of C in [VII] onto reproducible kernel Hilbert space:
$$Ψ ( C ) = [ \begin{matrix}κ ( {c}_{1} , {v}_{1} ) & … & κ ( {c}_{R} , {v}_{1} ) \\ … & … & … \\ κ ( {c}_{1} , {v}_{D} ) & … & κ ( {c}_{R} , {v}_{D} )\end{matrix} ] \qquad [ XI ]$$
where interpretation of Ψ(C) is equivalent to those of Ψ(A) in [IX],
using empirical kernel map for nonlinear mapping of D in [VIII] onto reproducible kernel Hilbert space:
$$Ψ ( D ) = [ \begin{matrix}κ ( {d}_{1} , {v}_{1} ) & … & κ ( {d}_{R} , {v}_{1} ) \\ … & … & … \\ κ ( {d}_{1} , {v}_{D} ) & … & κ ( {d}_{R} , {v}_{D} )\end{matrix} ] \qquad [ XII ]$$
where interpretation of Ψ(D) is equivalent to those of Ψ(A) in [IX],
applying sparseness and nonnegativity constrained matrix factorization (sNMF) algorithms to [IX], [X], [XI] and [XII] to obtain estimates of the pure components {sₘ}ₘ₌₁^{M} and some of their cross-products {sₘ_{₁}sₘ_{₂}}ₘ_{₁}ₘ_{₂₌₁}^{M}:
{ s̅ₘ^{A}}ₘ₌₁^{\hat{M}}=sNMF(Ψ(A)) [XIII]
{ s̅ₘ^{B}}ₘ₌₁^{\hat{M}}=sNMF(Ψ(B)) [XIV]
{ s̅ₘ^{C}}ₘ₌₁^{\hat{M}}=sNMF(Ψ(C)) [XV]
{ s̅ₘ^{D}}ₘ₌₁^{\hat{M}}=sNMF(Ψ(D)) [XVI]
where \hat{M} denotes overall number of components separated in [XIII], [XIV], [XV] and [XVI],
estimating further the pure components by correlating { s̅ₘ^{A}}ₘ₌₁^{\hat{M}} from [XIII], { s̅ₘ^{B}}ₘ₌₁^{\hat{M}} from [XIV], { s̅ₘ^{C}}ₘ₌₁^{\hat{M}} from [XV] and { s̅ₘ^{D}}ₘ₌₁ ^{M̅} from [XVI], with the components stored in the library composed of J reference compounds {sⱼ^{ref}}ⱼ₌₁^{J}:
$$\begin{matrix}{c}_{mj}^{A} = {argmax}_{j = 1 , … , J} \frac{〈 {\overline{s}}_{m}^{A} , {s}_{j}^{ref} 〉}{‖ {\overline{s}}_{m}^{A} ‖ ‖ {s}_{j}^{ref} ‖} m = 1 , … , \hat{M} , & [ XVII ] \\ {c}_{mj}^{B} = {argmax}_{j = 1 , … , J} \frac{〈 {\overline{s}}_{m}^{B} , {s}_{j}^{ref} 〉}{‖ {\overline{s}}_{m}^{B} ‖ ‖ {s}_{j}^{ref} ‖} m = 1 , … , \hat{M} , & [ XVIII ] \\ {c}_{mj}^{C} = {argmax}_{j = 1 , … , J} \frac{〈 {\overline{s}}_{m}^{C} , {s}_{j}^{ref} 〉}{‖ {\overline{s}}_{m}^{C} ‖ ‖ {s}_{j}^{ref} ‖} m = 1 , … , \hat{M} , & [ XIX ] \\ {c}_{mj}^{D} = {argmax}_{j = 1 , … , J} \frac{〈 {\overline{s}}_{m}^{D} , {s}_{j}^{ref} 〉}{‖ {\overline{s}}_{m}^{D} ‖ ‖ {s}_{j}^{ref} ‖} m = 1 , … , \hat{M} , & [ XX ]\end{matrix}$$
where [symbol] s̅ₘ,sⱼ^{ref} [symbol] , [symbol] s̅ₘ^{B},sⱼ^{ref} [symbol] , [symbol] s̅ₘ^{C},sⱼ^{ref} [symbol] and [symbol] s̅ₘ^{D},sⱼ^{ref} [symbol] denote the inner products respectively between s̅ₘ^{A}, s̅ₘ^{B}, s̅ₘ^{C}, s̅ₘ^{D} and sⱼ^{ref}. ∥ s̅ₘ^{A}∥, ∥ s̅ₘ^{B}∥, ∥ s̅ₘ^{C}∥, ∥ s̅ₘ^{D}∥ and ∥sⱼ^{ref}∥ denote, respectively, l₂-norm of s̅ₘ^{A}, s̅ₘ^{B}, s̅ₘ^{C}, s̅ₘ^{D} and sⱼ^{ref}.
assigning to each component in the library {sⱼ^{ref}}ⱼ₌₁^{J} components separated from [XIII], [XIV], [XV] and [XVI] that are indexed according to:
$$\begin{matrix}[ {c}^{A} , {m}_{A}^{*} ] = {argmax}_{m} {\{ {c}_{mj}^{A} \}}_{m = 1}^{{A}_{j}} & [ XXI ] \\ [ {c}^{B} , {m}_{B}^{*} ] = {argmax}_{m} {\{ {c}_{mj}^{B} \}}_{m = 1}^{{B}_{j}} & [ XXII ] \\ [ {c}^{C} , {m}_{C}^{*} ] = {argmax}_{m} {\{ {c}_{mj}^{C} \}}_{m = 1}^{{C}_{j}} & [ XXIII ] \\ [ {c}^{D} , {m}_{D}^{*} ] = {argmax}_{m} {\{ {c}_{mj}^{D} \}}_{m = 1}^{{D}_{j}} & [ XXIV ]\end{matrix}$$
where Aⱼ, Bⱼ, Cⱼ and Dⱼ respectively stand for number of separated components { s̅ₘ^{A}}ₘ₌₁^{\hat{M}}, { s̅ₘ^{B}}ₘ₌₁^{\hat{M}}, { s̅ₘ^{C}}ₘ₌₁^{\hat{M}} and { s̅ₘ^{D}}ₘ₌₁^{\hat{M}} associated respectively in [XVII], [XVIII], [XIX] and [XX] to reference component sⱼ^{ref}.
obtaining final estimates of the candidates for pure components {ŝⱼ}ⱼ₌₁^{J} according:
$$I = {argmax}_{A , B , C , D} ( {c}^{A} , {c}^{B} , {c}^{C} , {c}^{D} ) \\ {\hat{s}}_{j} = {\overline{s}}_{{m}_{I}^{*}} j = 1 , … , J and I ∈ \{ A , B , C , D \} . \qquad [ XXV ]$$
separated components { s̅ₘ^{A}}ₘ₌₁^{\hat{M}}, { s̅ₘ^{B}}ₘ₌₁^{\hat{M}}, { s̅ₘ^{C}}ₘ₌₁^{\hat{M}} and { s̅ₘ^{D}}ₘ₌₁^{\hat{M}} that are not assigned to the pure components from the library {ŝⱼ}ⱼ₌₁^{J}, are considered as candidates for new pure components.
presenting estimated candidates of pure components {ŝⱼ}ⱼ₌₁^{J} and candidates for new pure components from [XXV].

2. The method of claim 1, whereas exponential distribution is used to model probability density function of the amplitude: ƒ(sₘᵣ) in [III].

3. The method of claim 1, whereas robust principal component analysis, hard thresholding, soft thresholding and trimmed thresholding are used in [IV] to, respectively, yield [V], [VI], [VII] and [VIII].

4. The method of claim 1, where in empirical kernel maps [IX], [X], [XI] and [XII] a positive symmetric kernel function is shift invariant kernel: κ(aᵣ,v_{d})=κ(aᵣ−v_{d}). Preferably, κ(aᵣ,v_{d}) is Gaussian kernel: κ(aᵣ,v_{d})=exp(−∥aᵣ−v_{d}∥²/σ²) with variance σ²=1.

5. The method of claim 4, whereas basis {v_{d}}_{d=1}^{D} is obtained by some basis selection algorithm such that D≦R.

6. The method of claim 1, whereas number of components assumed to be present in [XIII], [XIV], [XV] and [XVI] is set to: \hat{M}=D.

7. The method of claim 1, whereas nonnegativity and sparseness constrained matrix factorization algorithm is applied in [XIII], [XIV], [XV] and [XVI] to separate \hat{M} components, respectively, from [IX], [X], [XI] and [XII].

8. The method of claim 1, whereas separated components are correlated with the reference components from the library in [XVII], [XVIII], [XIX] and [XX].

9. The method of claim 1, whereas indexes of components separated in [XIII], [XIV], [XV] and [XVI] to be assigned to pure components from the library are obtained according to [XXI], [XXII], [XXIII] and [XXIV].

10. The method of claim 1, whereas final estimates of the pure component candidates is performed according to [XXV].

11. The method of claim 1, whereas said method is applied to the identification of compounds in chemical synthesis, food quality inspection or pollution inspection.

12. The method of claim 1, whereas said method is applied to the identification of compounds from natural sources (microorganisms, plants and animals), metabolites and biomarkers present in biological fluids (urine, blood plasma, cerebrospinal fluid, saliva, amniotic fluid, bile, tears, etc) or tissue extracts.

13. The method of claim 1, whereas said method is applied to detection of pathologies (genetically determined diseases), detection of patients with predisposition for certain disease, monitoring the response of the organism to the action of pharmaceuticals, pathogens or toxic compounds (wars, natural or ecology disasters).
